(19) United States
(12) Patent Application Publication
(10) Pub. No.: US 2026/0174428 A1
(43) Pub. Date: Jun. 25, 2026

(54) SIZE ADAPTER ATTACHMENTS

(71) Applicant: Evalve, Inc., Santa Clara, CA (US)

(72) Inventors: Grant Glaze, San Francisco, CA (US); Chad J. Abunassar, Alameda, CA (US); Jessie A. Garcia, Newark, CA (US); Gabriel Gonzales, Milpitas, CA (US); Jill Mccoy, Los Altos, CA (US); Casey Barbarino, San Anselmo, CA (US); Santosh V. Prabhu, Sunnyvale, CA (US); Michael Zachary Chong, San Francisco, CA (US); Brandon Chu, San Francisco, CA (US); Scott Mosher, San Francisco, CA (US)

(73) Assignee: Evalve, Inc., Santa Clara, CA (US)

(21) Appl. No.: 19/127,606

(22) PCT Filed: Nov. 17, 2023
(86) PCT No.: PCT/US2023/080329

§ 371 Date: May 6, 2025

Related U.S. Application Data
(60) Provisional application No. 63/384,349, filed on Nov. 18, 2022.

(51) Int. Cl. A61B 17/04; A61B 17/064; A61B 17/122; A61F 2/24

(52) U.S. Cl. CPC A61B 17/0469; A61B 17/0644; A61B 17/122; A61F 2/246; A61F 2/2466; A61F 2220/0016; A61F 2220/0075

(57) ABSTRACT

A fixation device for fixation of heart valve leaflets includes a central assembly and an arm moveably coupled to the central assembly. The arm includes a body portion having a first end and a second end and a longitudinal axis defined therebetween. The body portion has opposing body lateral sides, each body lateral side extending between the first end and the second end. Further, the body portion has a body portion width defined between the opposing body lateral sides. The fixation device further includes a size adapter attached to the arm, the size adapter having a maximum undeformed arm lateral cross-dimension defined between outer lateral edges of the size adapter. The ratio between the body portion width and the maximum undeformed arm lateral cross-dimension is at least about 1:1.8 to about 1:2.2. The fixation device further includes at least one gripping element.

### CROSS-REFERENCE TO RELATED APPLICATIONS

This application claims the benefit of the filing date of U.S. Provisional Application No. 63/384,349, filed Nov. 18, 2022, the disclosure of which is hereby incorporated by reference.

### BACKGROUND OF THE INVENTION

### Field of Disclosed Subject Matter

The disclosed subject matter is directed to medical devices for the endovascular, percutaneous or minimally invasive surgical treatment of bodily tissues, such as tissue approximation or valve repair. More particularly, the present disclosure relates to repair of valves of the heart and venous valves.

Surgical repair of bodily tissues can involve tissue approximation and fastening of such tissues in the approximated arrangement. When repairing valves, tissue approximation includes coapting the leaflets of the valves in a therapeutic arrangement which can then be maintained by fastening or fixing the leaflets. Such coaptation can be used to treat regurgitation, which commonly occurs in the mitral valve and in the tricuspid valve.

Mitral valve regurgitation is characterized by retrograde flow from the left ventricle of a heart through an incompetent mitral valve into the left atrium. During a normal cycle of heart contraction (systole), the mitral valve acts as a check valve to prevent flow of oxygenated blood back into the left atrium. In this way, the oxygenated blood is pumped into the aorta through the aortic valve. Regurgitation of the mitral valve can significantly decrease the pumping efficiency of the heart, placing the patient at risk of severe, progressive heart failure.

Mitral valve regurgitation can result from a number of different mechanical defects in the mitral valve or the left ventricular wall. The valve leaflets, the valve chordae which connect the leaflets to the papillary muscles, the papillary muscles or the left ventricular wall can be damaged or otherwise dysfunctional. Commonly, the valve annulus can be damaged, dilated, or weakened limiting the ability of the mitral valve to close adequately against the high pressures of the left ventricle.

Tricuspid valve regurgitation has several causes. Functional tricuspid valve regurgitation (FTR) is characterized by structurally normal tricuspid valve leaflets that are nevertheless unable to properly coapt with one another to close properly due to other structural deformations of surrounding heart structures. For example, the right ventricle can become dilated as a result of pulmonary hypertension or an abnormal heart muscle condition (cardiomyopathy).

Other causes of tricuspid valve regurgitation are related to degenerative valves and/or defects of the tricuspid valve leaflets, tricuspid valve annulus, or other tricuspid valve structures. In some circumstances, tricuspid valve regurgitation is a result of infective endocarditis, blunt chest trauma, rheumatic fever, Marfan syndrome, carcinoid syndrome, improper placement of pacemaker leads, or congenital defects to the structure of the heart.

Tricuspid valve conditions are also often associated with problems related to the left side of the heart, such as mitral valve regurgitation. In particular, FTR is often associated with left heart pathologies, though the tricuspid valve is typically left untreated during left heart surgeries. Left heart pathologies such as mitral valve regurgitation and stenosis can induce pressure and volume overload in the right ventricle, which in turn can induce ventricle enlargement and tricuspid annular dilation. Though often relatively mild at the time of treatment of the left heart, this annular dilation of the tricuspid valve can be progressive and asymmetric, and FTR can become more severe as time goes on. Reoperation for repair of the tricuspid valve is often needed owing to the degenerative character of the pathology.

### DESCRIPTION OF RELATED ART

Treatments for mitral valve and tricuspid valve regurgitation rely on valve replacement or repair including leaflet and annulus remodeling, the latter generally referred to as valve annuloplasty. Another technique for valve repair, which relies on suturing adjacent segments of the opposed valve leaflets together is referred to as the “bow-tie” or “edge-to-edge” technique. Preferably, the use of devices and systems should not require open chest access and, rather, be capable of being performed either endovascularly, i.e., using devices, such as an interventional catheter, which are advanced to the heart from a point in the patient's vasculature remote from the heart. Furthermore, such devices and systems should allow for repositioning and optional removal of a fixation device (i.e., valve repair clip) prior to fixation to ensure optimal placement. Such devices and systems likewise can be useful for repair of tissues in the body other than heart valves.

### BRIEF SUMMARY OF THE DISCLOSURE

The purpose and advantages of the disclosed subject matter will be set forth in and apparent from the description that follows, as well as will be learned by practice of the disclosed subject matter. Additional advantages of the disclosed subject matter will be realized and attained by the methods and systems particularly pointed out in the written description and claims hereof, as well as from the appended drawings.

To achieve these and other advantages and in accordance with the purpose of the disclosed subject matter, as embodied and broadly described, the disclosed subject matter is directed to a fixation device for treating a patient.

In accordance with one aspect of the disclosed subject matter, a fixation device for fixation of leaflets of a heart valve including a central assembly and at least one arm moveably coupled to the central assembly. The at least one arm includes a body portion having a first end and a second end and a longitudinal axis defined therebetween, the second end being moveable between a closed position and an open position. The body portion has opposing body lateral sides, each body lateral side extending between the first end and the second end. Further, the body portion has a body portion width defined between the opposing body lateral sides. The fixation device further includes a size adapter attached to the at least one arm, the size adapter having a maximum undeformed arm lateral cross-dimension defined between outer lateral edges of the size adapter. The ratio between the body portion width and the maximum undeformed arm lateral cross-dimension is at least about 1:1.8 to about 1:2.2. The fixation device further includes at least one gripping element moveable relative to the at least one arm to capture a native leaflet therebetween.

In accordance with aspects of the disclosed subject matter, the fixation device can further include first and second nondeformable wing extension wherein each nondeformable wing extension extends laterally from a respective body lateral side. Each nondeformable wing extension can have a lateral outer edge. Further, a maximum wing width can be defined between the lateral outer edge of the first nondeformable wing extension and the lateral outer edge of the second nondeformable wing extension. The maximum wing width can be between about 1.40 and 1.60 of the body portion width in some examples.

Furthermore, the size adapter can be a first and second flexible attachment attached to the first and second nondeformable extension wing respectively, wherein each flexible attachment can comprise a flex portion and an attachment portion. The at least one arm can be in the maximum undeformed arm lateral cross-dimension when the flex portion is in an undeformed condition. The at least one arm can have a maximum deformed arm lateral cross-dimension when the flex portion is in a deformed condition. The ratio of the maximum deformed arm lateral cross-dimension to the maximum undeformed arm lateral cross-dimension can be at between 1:1 and 1:1.3 in various examples.

In accordance with another aspect of the disclosed subject matter, the first and second flexible attachments can be attached to the first and second nondeformable wing extensions, respectively, by at least one fastener selected from the group consisting of a suture, weld, solder, snap, bolt, clamp, rivet, crimp, and adhesive. Further, the at least one fastener can be at least one rivet. The first flexible attachment can be attached to the first nondeformable wing extension by at least two rivets, and the second flexible attachment can be attached to the second nondeformable wing extension by at least two rivets. The at least one arm can have a front surface and a back surface, wherein the front surface can faces the at least one gripping element when the at least one arm is in the closed position. The size adapter can be disposed on the back surface of the at least one arm. The size adapter can be curved or bent to the front surface. The first flexible attachment can be connected to the second flexible attachment by a span.

Additionally, the size adapter can include a material selected from the group consisting of: a plastic; a metal; and a composite. The size adapter can include a material selected from the group consisting of: copper-zinc-aluminum alloy; copper-aluminum-nickel alloy; nickel-titanium alloy; nickel-titanium platinum alloy; and nickel-titanium palladium alloy. The size adapter can be formed of Nitinol.

In accordance with another aspect of the disclosed subject matter, the size adapter can be a wire frame having a flexible structure. The wire frame can be secured to the at least one arm by at least one weld, wherein the at least one weld can be disposed proximate one of the opposing body lateral sides. The at least one weld can be disposed on a back surface of the arm, wherein the back surface of the body portion can face opposite the central assembly. The wire frame can include at least one wire loop. The wire frame can include at least two wire loops. The at least two wire loops can be configured to fold inwardly to fit within an inner diameter of an interventional catheter. The wire frame can include at least three wire loops. Furthermore, a tubing attachment can be welded to the at least one arm, and the wire frame can be inserted into the tubing attachment. The wire frame can include a first wire frame and a second wire frame, wherein the first wire frame is separate from the second wire frame. The wire frame can include a plurality of separate wires grouped together.

Additionally, the fixation device can further include an attachment wing extending laterally from the at least one arm, wherein the attachment wing and the at least one arm can be a single piece structure, and further wherein a portion of the wire frame can be attached to the attachment wing. The attachment wing can be formed of a sheet metal fold comprising sheet metal folded degrees around the portion of the wire frame. The attachment wing can be machined with a coining process. The attachment wing can include a machined slot sized for the portion of the wire frame. The attachment wing can be press fit around the portion of the wire frame. The attachment wing can be stamped around the portion of the wire frame.

Furthermore, a gusset member can be disposed between the body portion and the first nondeformable wing extension. The gusset member can include a hole, and the size adapter is attached to the hole. The body portion can include at least one notch, and the size adapter can be attached to the at least one notch by a snap fit. The at least one arm can include a strut member extending perpendicular to the longitudinal axis, and wherein the size adapter can be attached to the strut member. The size adapter can be attached to the at least one arm with a suture.

In accordance with another aspect of the disclosed subject matter, the size adapter can have a deformable portion and at least one reinforced portion, wherein the at least one reinforced portion can be less flexible than the deformable portion. The size adapter can have a second end disposed proximate the second end of the at least one arm, and wherein the second end of the size adapter can include a radiused corner. The size adapter can have a first end disposed closer to the first end of the at least one arm than the second end of the at least one arm, and wherein the first end of the size adapter can include a radiused corner. The size adapter can extend beyond the second end of the body portion. The size adapter can include at least one opening aligned with the second end of the body portion. The size adapter can include a deformable frame having at least one deformable flex portion, wherein the at least one deformable flex portion includes a wire element defining an opening therethrough in plan view. The wire element can extend beyond the second end of the body portion. The deformable frame can be attached to the at least one arm by a molded structure. The molded structure can extend beyond the second end of the body portion.

Additionally, the size adapter can be configured to pivot relative to the at least one arm at a pin connection. The size adapter can include at least one spring configured to bias the size adapter towards the maximum undeformed arm lateral cross-dimension. The size adapter can include an elongate member pivotally attached at a first end to the at least one arm and extending to a second end that is a free end wherein the at least one spring can be configured to bias the second end laterally outwardly from the at least one arm. The size adapter can include a first section and a second section with springs disposed between the first section and the second section configured to separate the first section and the second section from each other towards the maximum undeformed arm lateral cross-dimension. The size adapter can include an expandable member configured to have more fluid in an expanded condition than in an unexpanded condition. The expandable section can be a fillable section or an absorbable section. The size adapter can include a radiopaque marker.

### BRIEF DESCRIPTION OF THE FIGURES

FIG. 1 is a perspective view of an exemplary embodiment of a fixation device for use in accordance with the disclosed subject matter.

FIG. 2 is a front view of the fixation device of FIG. 1, wherein optional arms of greater length are depicted with dashed lines.

FIGS. 3A-3C are front views of the fixation device of FIG. 1 at various positions, wherein optional arms of greater length are depicted with dashed lines.

FIG. 4A is a front schematic view of the fixation device of FIG. 1 having leaflets captured therein.

FIG. 4B is a side view of the fixation device of FIG. 1 to schematically depict a contact patch area.

FIG. 5 is a plan view of an exemplary embodiment of an arm in accordance with the disclosed subject matter, the arm having features and characteristics being suitable for use in any fixation device of the disclosure.

FIG. 6 is a plan view of an alternative embodiment of an arm having nondeformable wing extensions, the arm having features and characteristics being suitable for use in any fixation device of the disclosure.

FIG. 7 is back view of the arm of FIG. 6 having a size adapter in an undeformed condition.

FIG. 8 is a back view of the arm of FIG. 6 having a size adapter in a deformed condition.

FIG. 9 is an end view of the size adapter of FIG. 6 having a curved shape suitable for integration with any size adapter of the disclosure.

FIG. 10 is an end view of the size adapter of FIG. 6 having a bent shape suitable for integration with any size adapter of the disclosure.

FIG. 11 is a back view of an alternative embodiment of a size adapter having a span element suitable for integration with any size adapter of the disclosure.

FIGS. 12A-12B are plan view of alternative embodiments of a flexible attachment suitable for use with all examples of the disclosure.

FIG. 13 is a back view of an arm and an alternative embodiment of a size adapter having reduce sized flexible attachments, aspects of which are suitable for incorporating into all examples of the disclosure.

FIG. 14 is a back view of an arm and an alternative embodiment of a size adapter having a wire loop, aspects of which are suitable for incorporating into all examples of the disclosure.

FIG. 15 is a back view of an arm and an alternative embodiment of a size adapter having two wire loops, aspects of which are suitable for incorporating into all examples of the disclosure.

FIG. 16 is the arm and size adapter of size adapter of FIG. 15 and a schematic of the size adapter folding into an interventional catheter.

FIG. 17 is an arm and an alternative embodiment of a size adapter having two wire loops and a welded attachment, aspects of which are suitable for incorporating into all examples of the disclosure.

FIG. 18 is an arm and an alternative embodiment of a size adapter having three wire loops and a welded attachment, aspects of which are suitable for incorporating into all examples of the disclosure.

FIG. 19 is an arm and an alternative embodiment of a size adapter attached to the arm via a tubing attachment, aspects of which are suitable for incorporating into all examples of the disclosure.

FIG. 20 is an arm and an alternative embodiment of a size adapter having a first and second wire frame welded to the arm, aspects of which are suitable for incorporating into all examples of the disclosure.

FIG. 21 is an arm and an alternative embodiment of a size adapter having a first and second wire frame attached to the arm via a tubing attachment, aspects of which are suitable for incorporating into all examples of the disclosure.

FIG. 22 is an arm and an alternative embodiment of a size adapter having a plurality of separate wires welded to the arm, aspects of which are suitable for incorporating into all examples of the disclosure.

FIG. 23 is an arm and an alternative embodiment of a size adapter having a plurality of separate wires attached to the arm via a tubing attachment, aspects of which are suitable for incorporating into all examples of the disclosure.

FIG. 24A is an arm having a sheet metal fold attachment wing, aspects of which are suitable for incorporating into other examples of the disclosure.

FIG. 24B is the arm and attachment wing of FIG. 24B further including a slot, aspects of which are suitable for incorporating into other examples of the disclosure.

FIG. 25 is the arm of FIG. 6 further including a gusset, aspects of which are suitable for incorporating into other examples of the disclosure.

FIG. 26A-26B is an alternative embodiment of an arm including notch attachment portions, aspects of which are suitable for incorporating into all examples of the disclosure.

FIG. 27A-27B is the arm of FIGS. 26A-26B including an alternative embodiment of a size adapter, aspects of which are suitable for incorporating into all examples of the disclosure.

FIG. 28 is a plan view of a portion of an arm having an alternative embodiment of the size adapter attached thereto, aspects of which are suitable for incorporating into all examples of the disclosure.

FIG. 29 is an end view of the portion of the arm and the size adapter depicted in FIG. 28.

FIG. 30 is an end view of a first arm and a first size adapter, and a second arm and second size adapter within an interventional catheter, aspects of which are suitable for incorporating into all examples of the disclosure.

FIG. 31 is an end view of an alternative embodiment of a first arm and a first size adapter overlapping with a second arm and second size adapter, aspects of which are suitable for incorporating into all examples of the disclosure.

FIG. 32 is a perspective view of an alternative embodiment of a size adapter having a deformable portion and a reinforced portion, aspects of which are suitable for incorporating into all examples of the disclosure.

FIG. 33 is a perspective view of an arm having an alternative embodiment of a size adapter having radiused corners, aspects of which are suitable for incorporating into all examples of the disclosure.

FIG. 34 is a plan view of the alternative embodiment of the size adapter depicted in FIG. 33.

FIG. 35 is a plan view of the alternative embodiment of the size adapter without radiused corners, aspects of which are suitable for incorporating into all examples of the disclosure.

FIGS. 36 and 37 are end views of the alternative embodiment of the size adapter depicted in FIG. 33.

FIGS. 38-39 are back plan views of alternative embodiments of a size adapter with deformable frame, aspects of which are suitable for incorporating into all examples of the disclosure.

FIG. 40 is a front perspective view of the alternative embodiment of a size adapter depicted in FIGS. 38-39 and further having a fastener.

FIG. 41 is a back perspective view of an alternative embodiment of a size adapter with an alternative deformable frame, aspects of which are suitable for incorporating into all examples of the disclosure.

FIG. 42 is a front plan view of the alternative embodiment of a size adapter with the alternative wire frame depicted in FIG. 41 and further having a fastener.

FIG. 43 is a perspective view of an alternative embodiment of a size adapter having a deformable frame, aspects of which are suitable for incorporating into all examples of the disclosure.

FIG. 44 is a front view of the alternative embodiment of a size adapter with the alternative wire frame depicted in FIG. 43 with a force applied thereto.

FIG. 45 is a back perspective view of an alternative embodiment of a size adapter having a wire frame and a molded structure, aspects of which are suitable for incorporating into all examples of the disclosure.

FIGS. 46A-46C are exemplary size adaptor geometries from FIG. 45 shown in a side profile view.

FIG. 47 is a perspective view of an arm having an alternative embodiment of a size adapter having a rivet attachment, aspects of which are suitable for incorporating into all examples of the disclosure.

FIGS. 48A and 48B are plan views of an arm having an alternative embodiment of a size adapter having a pivoting spring deployment mechanism, aspects of which are suitable for incorporating into all examples of the disclosure.

FIGS. 49A and 49B are plan views of an arm having an alternative embodiment of a size adapter having a lateral translation spring deployment mechanism, aspects of which are suitable for incorporating into all examples of the disclosure.

FIGS. 50A and 50B are plan views of an arm having an alternative embodiment of a size adapter having a pivoting wire spring and track deployment mechanism, aspects of which are suitable for incorporating into all examples of the disclosure.

FIGS. 51A and 51B are plan views of an arm and a cover having an alternative embodiment of a size adapter having an expandable member, aspects of which are suitable for incorporating into all examples of the disclosure.

FIG. 52A is a front plan view of a size adapter having a radiopaque marker, aspects of which are suitable for incorporating into all examples of the disclosure.

FIG. 52B is a front view of a size adapter having a radiopaque marker, aspects of which are suitable for incorporating into all examples of the disclosure.

FIG. 53 is a top view of an interventional catheter assembly in accordance with the disclosed subject matter, aspects of which are suitable for incorporating into all examples of the disclosure.

### DETAILED DESCRIPTION

Reference will now be made in detail to the various exemplary embodiments of the disclosed subject matter, exemplary embodiments of which are illustrated in the accompanying drawings.

The fixation device for use with the disclosed subject matter provides an edge-to-edge transcatheter valve repair option for patients having various conditions, including regurgitant mitral valves or tricuspid valves. Transcatheter (e.g., trans-septal) edge-to-edge valve repair has been established using a fixation device, such as the MitraClip® Transcatheter Mitral Valve Repair device and the TriClip® Transcatheter Tricuspid Valve Repair device. These fixation devices generally are configured to capture and secure opposing native leaflets using two types of leaflet contacting elements. The first element is a sub-valvular arm (also known as a distal element or fixation element) to contact the ventricular side of a native leaflet to be grasped. With the arm positioned underneath to stabilize the native leaflet in a beating heart, a second gripping element (e.g., a proximal element) can be lowered or moved toward the arm and into contact with the atrial side of the native leaflet to capture the leaflet therebetween. Once each native leaflet is captured by a respective arm and gripping element, the fixation device can be closed by raising or moving the arms toward a center of the fixation device such that the leaflets are brought into coaptation, which results in a reduction in valvular regurgitation during ventricular systole. Furthermore, a covering can be provided on the arms and/or gripping elements to facilitate tissue ingrowth with the captured leaflets.

Additional details of exemplary fixation devices in accordance with the disclosed subject matter are set forth below. Furthermore, a number of patents and publications disclose additional details and aspects of such fixation devices and related operations. See for example, U.S. Pat. No. 7,226,467 to Lucatero et al.; U.S. Pat. No. 7,563,267 to Goldfarb et al.; U.S. Pat. No. 7,655,015 to Goldfarb et al.; U.S. Pat. No. 7,736,388 to Goldfarb et al.; U.S. Pat. No. 7,811,296 to Goldfarb et al.; U.S. Pat. No. 8,057,493 to Goldfarb et al.; U.S. Pat. No. 8,303,608 to Goldfarb et al.; U.S. Pat. No. 8,500,761 to Goldfarb et al.; U.S. Pat. No. 8,734,505 to Goldfarb et al.; U.S. Pat. No. 8,740,920 to Goldfarb et al.; U.S. Pat. No. 9,510,829 to Goldfarb et al.; U.S. Pat. No. 7,635,329 to Goldfarb et al.; U.S. Pat. No. 11,065,119 to Abunassar et al., U.S. Patent Application Publication No. 2017/0042546 to Goldfarb et al.; U.S. Patent Application Publication No. 2017/0239048 to Goldfarb et al.; U.S. Patent Application Publication No. 2021/0186698 to Abunassar et al.; and U.S. Provisional Patent Application No. 63/182,167 filed Apr. 30, 2021, the entirety of the contents of each of these patents and published applications is incorporated herein by reference.

In grasping tissue and leaflet capture for mitral and tricuspid valve disease, certain patient conditions and anatomies, such as those associated with larger dynamic gaps between leaflet tips, can create challenges for capture. As such, there is an opportunity for a fixation device capable of bridging larger gaps, such as in functional mitral regurgitation (FMR) and functional tricuspid regurgitation (FTR), while also providing more reliable leaflet capture, for example in cases of dynamic, chaotic, or overly severe degenerative mitral regurgitation (DMR), such as in cases of Barlow's Syndrome, and severe degenerative tricuspid regurgitation (DTR), such as in the case of Ebstein's Anomaly. Particularly, the size and configuration of the arm of the fixation device can significantly improve performance. However, such modifications can be configured to account for numerous factors to produce desired clinical benefit and still be deliverable transvascularly through an interventional catheter. For example, a typical interventional catheter size for delivery can have an inner diameter of about 0.220 inch or less. Furthermore, when positioned within a patient, the interventional catheter defines a tortious path through which the fixation device can be delivered. As such, the fixation device can be configured to be capable of such delivery through the corresponding bends and turns of the interventional catheter.

Additionally, and as previously noted, the fixation device can be configured to capture or grasp a leaflet between the arm and the gripping element. When in the closed position, it can facilitate further capture of adjacent leaflets positioned between two arms in the final implanted condition. Such capture can be a function of a contact patch area of the leaflets as defined by the width, length, and configuration of the arms and corresponding size adapters. An increased contact patch area can provide a more uniformed distribution of stresses in the grasped leaflets and can increase the radius of curvature of the grasped leaflet, which can help to keep the grasped leaflet intact. Hence, increasing arm or size adapter width and/or length can increase contact patch area and corresponding capture. In this manner, and in accordance with the disclosed subject matter, the fixation device can include a size adapter attached to the arm having a desired size adapter width, length, and configuration to provide a desired contact patch area, while still being capable of delivery.

Generally, and as set forth in greater detail below, the disclosed subject matter provided herein includes, a fixation device for fixation of leaflets of a heart valve including a central assembly and at least one arm moveably coupled to the central assembly. The at least one arm includes a body portion having a first end and a second end and a longitudinal axis defined therebetween, the second end being moveable between a closed position and an open position. The body portion has opposing body lateral sides, each body lateral side extending between the first end and the second end. Further, the body portion has a body portion width defined between the opposing body lateral sides. The fixation device further includes a size adapter attached to the at least one arm, the size adapter having a maximum undeformed arm lateral cross-dimension defined between outer lateral edges of the size adapter. In various examples, the ratio between the body portion width and the maximum undeformed arm lateral cross-dimension is at least about 1:1.8 to about 1:2.2. The fixation device further includes at least one gripping element moveable relative to the at least one arm to capture a native leaflet therebetween.

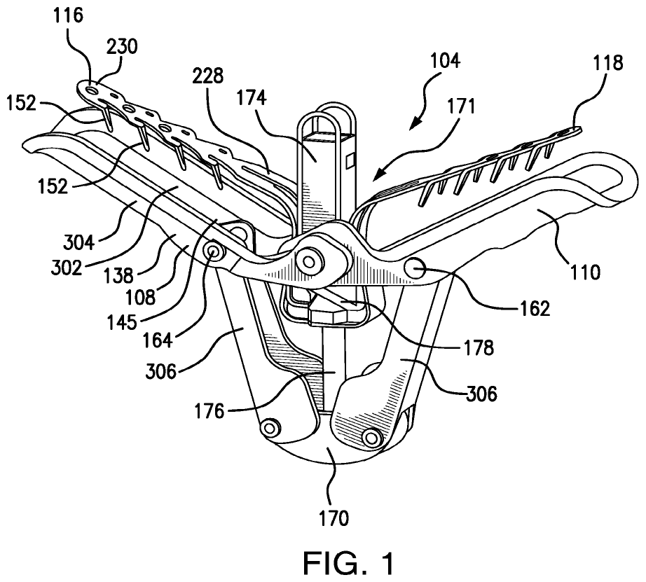

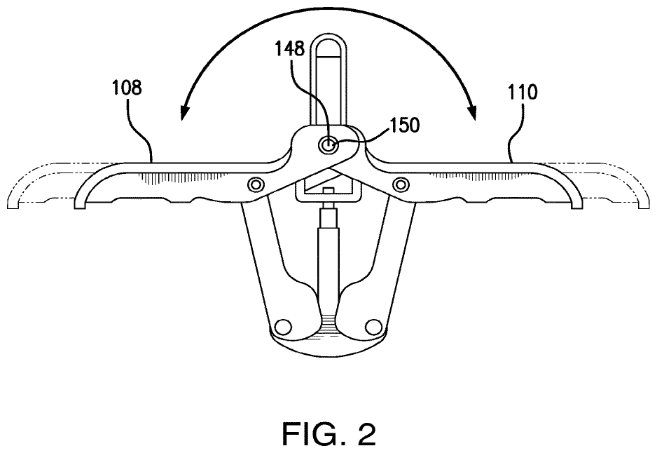

Referring to FIGS. 1-2A for the purpose of illustration and not limitation, an example of a fixation device 104 for fixation of leaflets of a heart valve is disclosed herein. The fixation device as embodied herein includes a central assembly 171. The central assembly 171 can include various central components for operation and release of the fixation device, for example, a coupling member 174 as described further in the disclosures of the patents and applications incorporated in their entirety by reference herein. The fixation device as depicted further includes at least one arm 108 moveably coupled relative to the central assembly 171. As shown, the fixation device 104 can further include a second arm 110 moveably coupled relative to the central assembly 171. For purpose of understanding and reference only, FIGS. 1-6 depict the arms without having attached thereto a size adapter 200 of the disclosed subject matter.

With reference to FIG. 2, for illustration and not limitation, each arm 108, 110 can be rotatable or moved about a respective axis point 148, 150 between closed, open and inverted positions, as well as any position therebetween. Furthermore, the arms 108, 110 can be selected from a range of suitable lengths, wherein the appropriate length can be selected by the physician or health care provider after inspection of a patient. For purpose of comparison, a first length of each arm 108, 110 is depicted in FIG. 2 in solid lines, and a second longer length of each arm of the disclosed subject matter is depicted in dashed lines. Each arm depicted in solid lines can be an entirely separate arm with a different length as compared to the corresponding arm depicted in dashed lines.

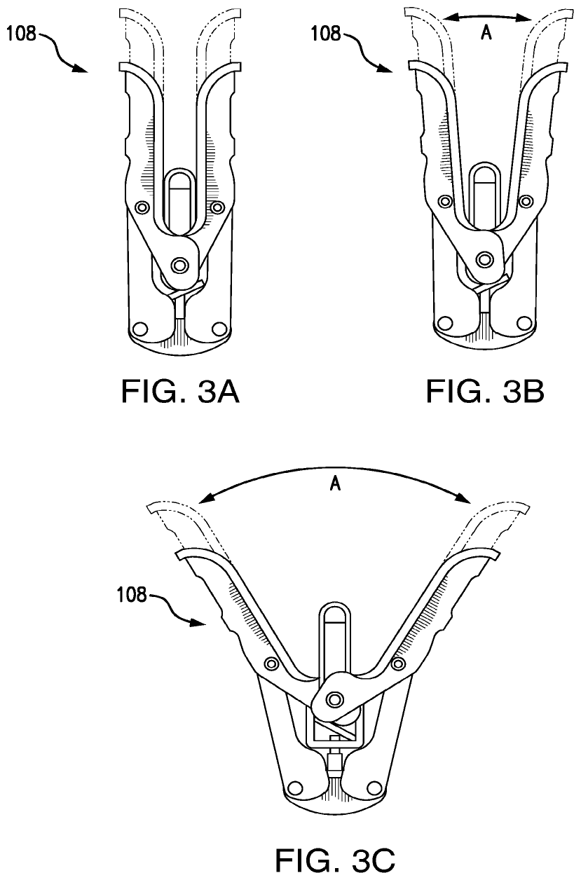

As depicted herein in FIGS. 3A-3C, various positions of the fixation device 104 are depicted for purpose of illustration and not limitation. Arms 108 of longer length are illustrated in dashed lines for comparison to shorter arms. In FIG. 3A, the fixation device is in the closed position, wherein the arms 108 are positioned axially in alignment, e.g., vertically or nearly vertically as shown. FIGS. 3B and 3C illustrate the arms positioned with an angle A between each other. In FIG. 3B, angle A is about 10 degrees and in FIG. 3C angle A is about 60 degrees. As disclosed herein, the fixation device is in the closed position when angle A is about 30 degrees or less, although another angle may result when leaflets of greater thickness are captured therebetween. Although not depicted, the arms can continue to open until angle A exceeds 180 degrees, e.g., inverted.

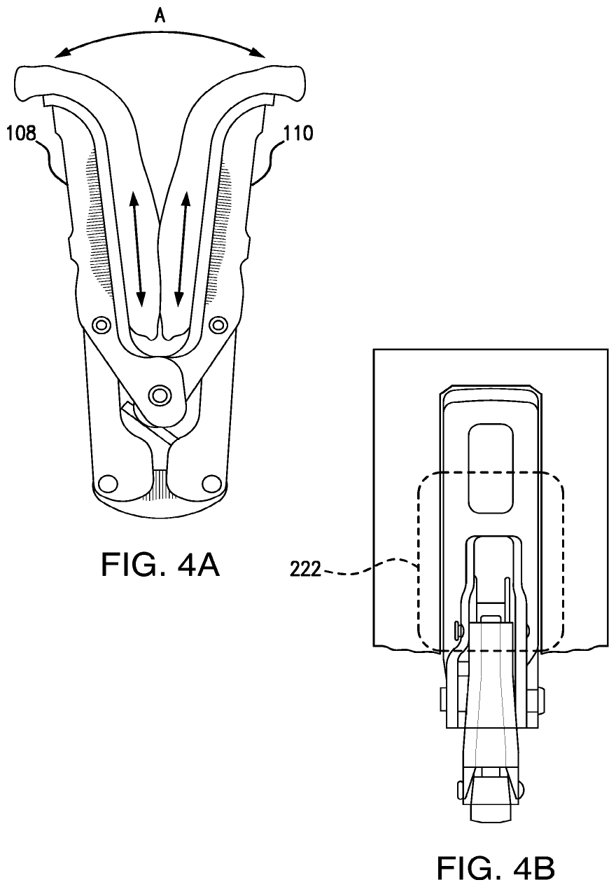

As previously noted generally, and as set forth in further detail below, a native leaflet can be captured between each arm and a respective gripping element. Each arm can then be moved toward its closed position. In this manner, adjacent leaflets can further be captured between two arms in the closed position. For example, and for illustration only, FIGS. 4A-4B show the fixation device 104 depicted with the arms 108, 110 at an angle A of about 20 to 30 degrees with two leaflets captured therebetween, wherein each leaflet is captured between an arm and a respective gripping element (wherein the gripping element is not shown for clarity). As illustrated in FIG. 4B, a contact patch 222 depicted in dashed lines is defined by the area of tissue contact between the arms and corresponding size adapters. The contact patch area 222 represents a tissue-to-tissue contact patch area defined by the area of a leaflet in contact with a counterpart leaflet. As previously noted, FIG. 4B depicts a representative contact patch area when each arm does not include a size adapter attached thereto and the arms of the fixation device are at an angle A of about 20 to 30 degrees. The angle A can affect the contact patch area 222 wherein a reduced angle A can increase the contact patch area 222, and likewise an increased angle A can decrease the contact patch area 222.

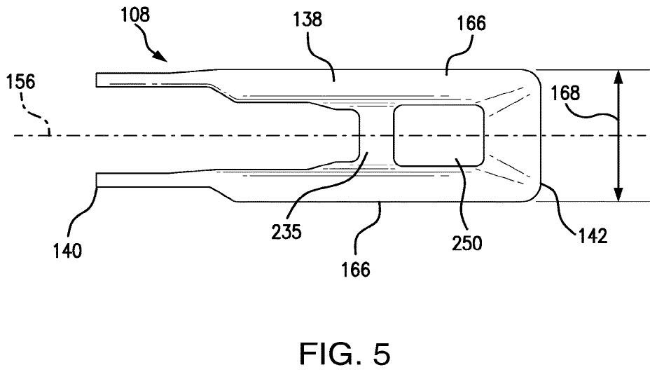

With additional reference to FIG. 5, the at least one arm 108 includes a body portion 138 having a first end 140 and a second end 142 and a longitudinal axis 156 defined therebetween. The second end 142 is moveable between a closed position and an open position. The body portion 138 has opposing body lateral sides 166, and each body lateral side 166 extends between the first end 140 and the second end 142. The body portion 138 has a body portion width 168 defined between the opposing body lateral sides 166. The at least one arm 108 can include a strut member 235 extending perpendicular to the longitudinal axis 156. The at least one arm can include an aperture 250 and the strut 235 can define one side of the aperture 250.

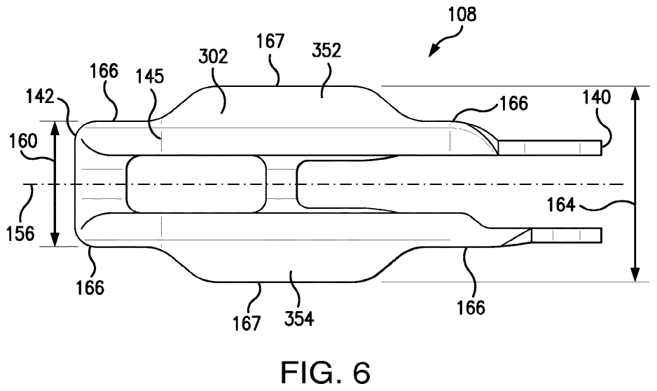

With further reference to FIG. 6, the fixation device 104 can further include first and second nondeformable wing extensions 352, 354, each nondeformable wing extension 352, 354 can extend laterally from a respective body lateral side 166. Each nondeformable wing extension 352, 354 can have a lateral outer edge 167. A maximum wing width 164 can be defined between the lateral outer edge 167 of the first nondeformable wing extension 352 and the lateral outer edge 167 of the second nondeformable wing extension 354. The maximum wing width 164 can be between about 1.40 and 1.60 of the body portion width 168. The maximum wing width 164 can preferably be between about 1.50 of the body portion width 168.

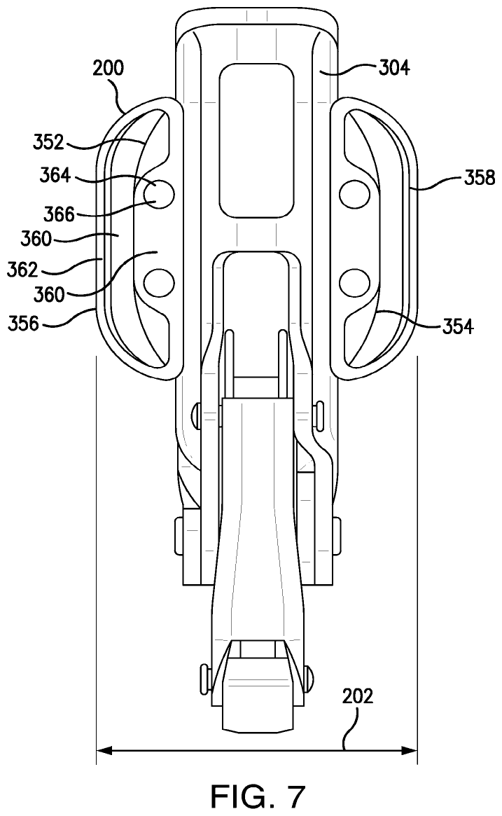

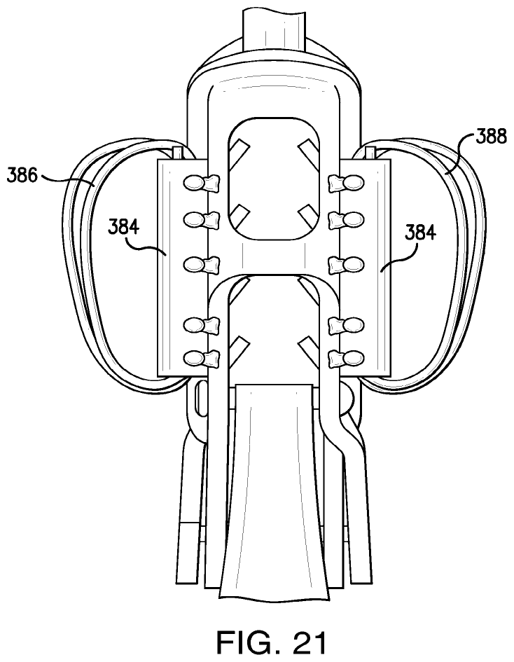

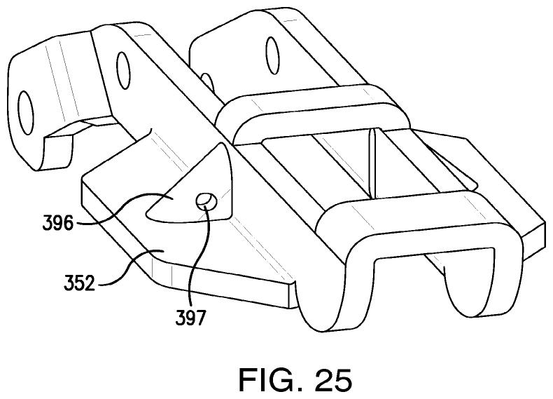

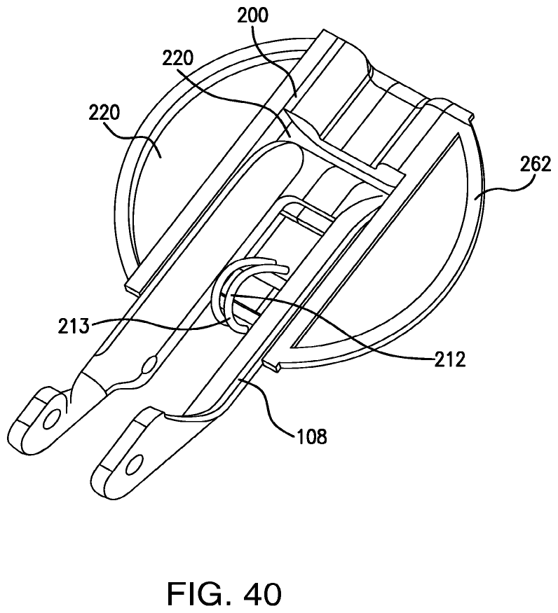

With further reference to FIGS. 7-52, various examples of the fixation device further include a size adapter 200 attached to the at least one arm 108. In accordance with various examples of the disclosed subject matter, the size adapter 200 can include a material selected from the group consisting of: a plastic; a metal; and a composite. The size adapter 200 can, for example, include a material selected from the group consisting of: copper-zinc-aluminum alloy; copper-aluminum-nickel alloy; nickel-titanium alloy; nickel-titanium platinum alloy; and nickel-titanium palladium alloy. The size adapter 200 can be formed of Nitinol and can be a single piece structure in various examples. During manufacturing, the size adapter 200 can be shape set, 3D printed direct metal laser sintered (DMLS), machined, and/or laser-cut, for example.

The size adapter 200 has a maximum undeformed arm lateral cross-dimension 202 defined between outer lateral edges of the size adapter 200. The ratio between the body portion width 168 and the maximum undeformed arm lateral cross-dimension 202 is at least at least about 1:1.8 to about 1:2.2, with a preferable ratio of 1:2.0, although other suitable ratios can be used. For example, is some embodiments the ratio between the body portion 138 width and the maximum undeformed arm lateral cross-dimension 202 is about 1:2. The repair device in accordance with the disclosed subject matter can have flexibility in at one least portion of the device in some examples. As disclosed herein, one manner in which flexibility can be achieved is by using multiple materials combining a rigid body of an arm with flexible attachments.

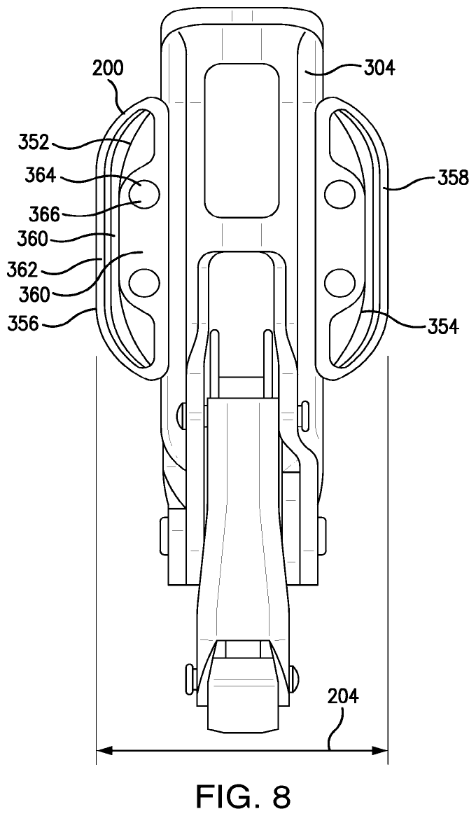

For example, and as shown in FIGS. 7-8, the size adapter 200 can be a first and second flexible attachment 356, 358 attached to the first and second nondeformable extension wing 352, 354, respectively, wherein each flexible attachment 356, 358 can comprise a flex portion 362 and an attachment portion 360. As shown in FIG. 7, the at least one arm 108 can be in the maximum undeformed arm lateral cross-dimension 202 when the flex portion 362 is in an undeformed condition. As shown in FIG. 8, the at least one arm 108 can have a maximum deformed arm lateral cross-dimension 204 when the flex portion 362 is in a deformed condition. In other words, the flex portion 362 of each flexible attachment 326, 358 is moveable inwardly toward the longitudinal axis 156 from a nondeformed condition to a deformed condition to reduce the lateral cross-dimension of arm 108 so that it can occupy a smaller footprint within an interventional catheter. Each flex portion 362 may be biased toward the nondeformed condition and, when compressed to the deformed condition, may increase in length in conjunction with the reduction in width according to one example. It should be noted that the reduction in width may be uniform, in some examples, along an entire length of each flexible attachment 356, 358. The ratio of the maximum deformed arm lateral cross-dimension 204 to the maximum undeformed arm lateral cross-dimension 202 can be between 1:1 and 1.3:1, although other suitable ratios can be used.

With continued reference in particular to FIG. 7, the first and second flexible attachments 356, 358 can be attached to the first and second nondeformable wing extensions 352, 354, respectively, by at least one fastener 364 such as a suture, weld, solder, snap, bolt, clamp, rivet, crimp, and/or adhesive, for example. The at least one fastener 364 can be at least one rivet 366 in various examples. The first flexible attachment 356 can be attached to the first nondeformable wing extension 352 by at least two rivets 366 in one example, and the second flexible attachment 354 can be attached to the second nondeformable wing extension 354 by at least two rivets 366 in an example. Example types of rivets that used may be used include cold formed tubular rivets, semi-tubular rivets, or solid rivets (of round, oval, square, rectangular, or other cross-section geometries). Alternatively, attachments may be soldered or welded joints, melted or adhesive connections, stitched or tied connections, molded or fused attachments, or any combination thereof. As best shown in FIGS. 7 and 8, each flex portion 362 may form a deformable closed loop extending from attachment portion 360. In one example, flex portion 362 may extend proximally and distally from attachment portion 360 and then outwardly to form the closed loop such that a gap or opening is formed between attachment portion 360 and a lateral extent of flex portion 362. Additionally, the closed loop defined by each flex portion 362 and corresponding attachment portion 360 may be oblong such that it is longer than it is wide, as best shown in FIGS. 12A and 12B.

Furthermore, the at least one arm 108 can have a front surface 302, as shown in the example of FIG. 6, and a back surface 304, as shown in the example of FIG. 7, wherein the front surface 302 can face the at least one gripping element 116 when the at least one arm 108 is in the closed position. As shown, the size adapter 200 can be disposed on the back surface 304 of the at least one arm 108 in various examples. Although not shown, in an alternative embodiment the size adapter can likewise be disposed on the front surface 302 of the at least one arm 108. It is understood that the features of FIG. 7 and various other figures throughout this application only depict a single arm 108 and a single corresponding size adapter 200 for purpose of clarity. That is, the features described herein can apply similarly to one or more additional arms and corresponding size adapters.

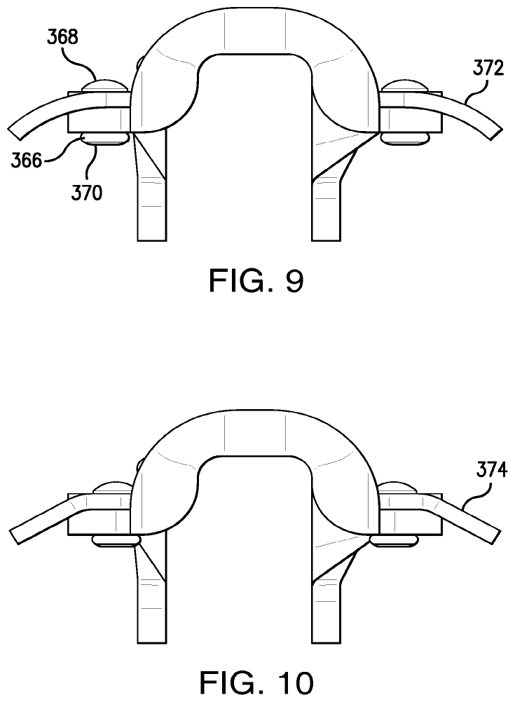

Referring now in addition to FIGS. 9 and 10, the size adapter 200 can be curved or bent to the front surface according to various examples. An exemplary embodiment of a curved size adapter 372 is shown in FIG. 9. The rivet 366 can include a factory (machined geometry) head 368 and a deformed head 370 in some examples. An exemplary embodiment of a bent size adapter 374 is shown in FIG. 10. The constant curvature geometry shown in the examples of FIG. 9 is amenable to parts being cut from a tube without requiring additional shape setting operations. The focused curvature shown in FIG. 10 enables a fully flat rivet bearing surface while enabling manufacturing from a flat sheet or strip that would require shape setting operations. Deformed head 370 may be favorably formed with a low profile such that focal contact stresses with the leaflets being repaired are minimized.

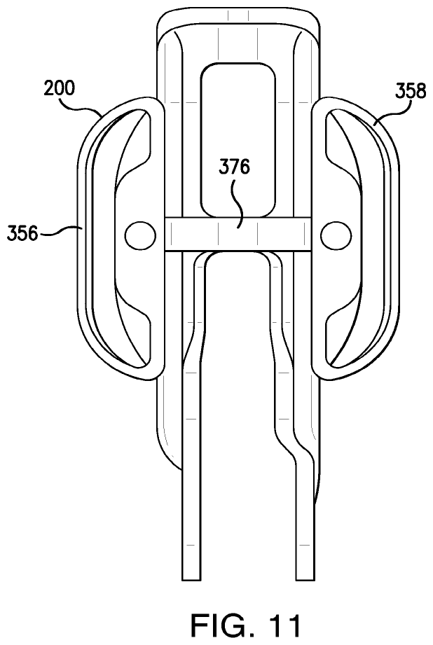

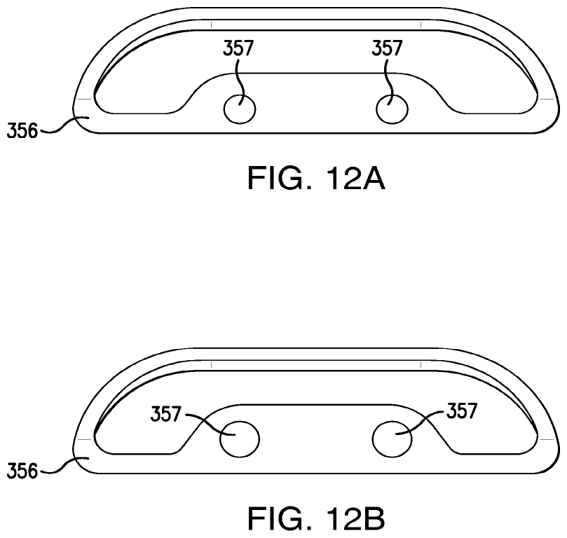

Referring now in addition to FIG. 11, the first flexible attachment 356 can be connected to the second flexible attachment 358 by a span 376 which extends laterally across the longitudinal axis of 156 of arm 108 in one example. The first flexible attachment 356, the second flexible attachment 358, and the span 376 can be formed of a single piece structure (i.e., a monolithic structure) in one example. The span 376 can have a wrap-around shape can be used to align the parts of the size adapter 200 and reduce the number of parts of the fixation device in some examples. Moreover, the span 376 can provide stiffness in the compression direction in some examples.

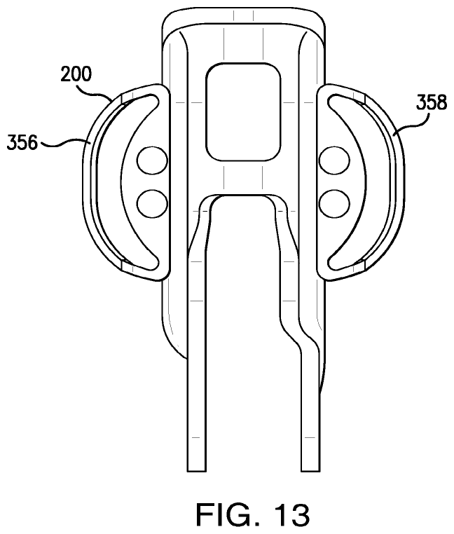

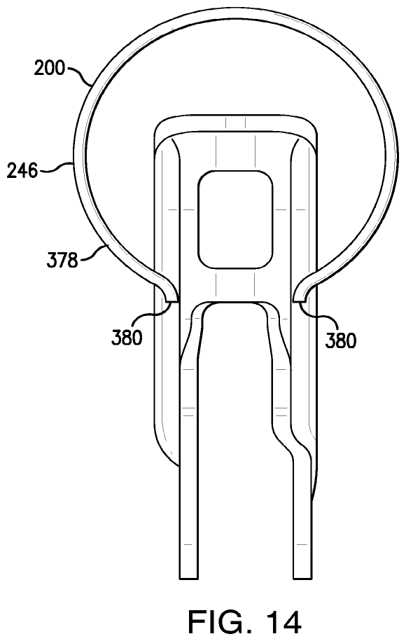

Referring now in addition to FIGS. 12A-12B, which show an exemplary first flexible attachment 356 of in which flex portion 362 has a thickness within the range of 0.004 inch to 0.014 inch, although other suitable thicknesses can be used. The overall length of flexible attachment 356 may make up 50% to 80% of a length of rigid arm 108 in contact with leaflets, although other suitable length dimensions can be used. The flexible attachment component lengths may fall within a range of 0.16 inch to 0.28 inch, although other suitable length dimensions can be used. When flexible attachments 356, 358 are attached to arm 108, flexible attachments 356, 358 may not extend beyond a second end 142 of arm such that flexible attachments 356 and 358 may only increase the effective width of arm 108 and not its length unlike other embodiments discussed below. In one example, he flexible attachment 356 can have holes 357 sized for a rivet to be inserted therein. The holes 257 can have a diameter between 0.013 inch and 0.025 inch, and preferably about 0.016 inch or about 0.021 inch, although other suitable diameters can be used. During manufacturing, the flexible attachment 356 can be laser cut, microblasted, etched, and electropolished in some examples. FIG. 13 shows a size adapter 200 in accordance with the disclosed subject matter having alternative dimensions.

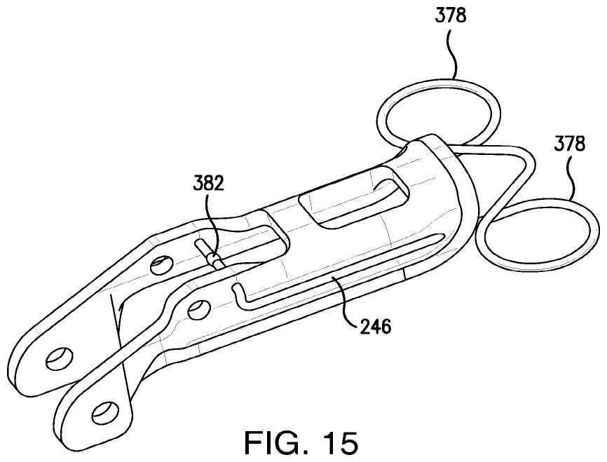

Referring in addition to FIGS. 14-23, the example, size adapter 200 can be a wire frame 246 having a flexible structure. The wire frame 246 can, for example, be secured to the at least one arm 108 by at least one weld 380, wherein the at least one weld 380 can be disposed proximate one of the opposing body lateral sides 166. The at least one weld 380 can, for example, be disposed on a back surface 304 of the arm 108, wherein the back surface 304 of the body portion 138 can face opposite the central assembly 171. In the embodiment depicted, opposed ends of wire frame may be fixedly secured to arm 108 at first and second connection locations via welds 380. In this regard, opposed ends of wire frame 246 are fixedly and immovably secured in this example to arm 108. In another example, shown in FIG. 15, wire frame 246 extends through openings in arm 108 such that the opposed ends of wire frame 246 meet within arm 108 and are joined together, such as via a weld 382. However, other exemplary mechanisms may be used to fixedly secure wire frame 246 to arm 108, such as via a swage, crimp, and press fit, for example. The wire frame 246 can extend the length of the fixation device 104 by any length, such as about 5/16 inch. The wire frame 246 can be formed of rounded, sheet, or strip wire in some examples. The wire cross section may be round, square, rectangular, triangular, hexagon, oval, or strip, for example. Flexible attachment may be cut out of a sheet and/or formed to produce the desirable shape. Flexible attachment may also be made via 3D printing.

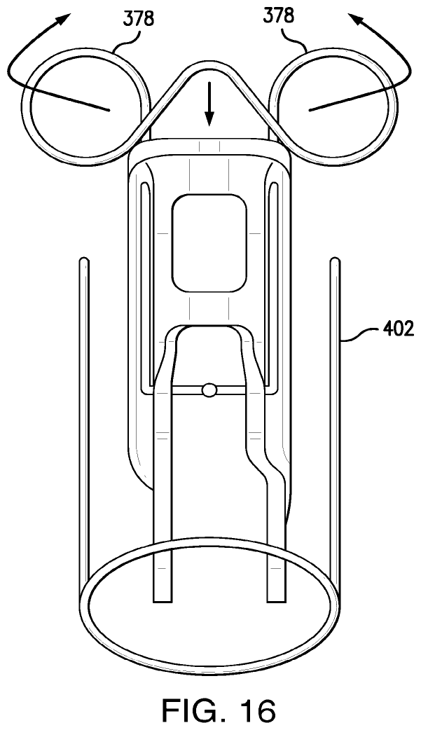

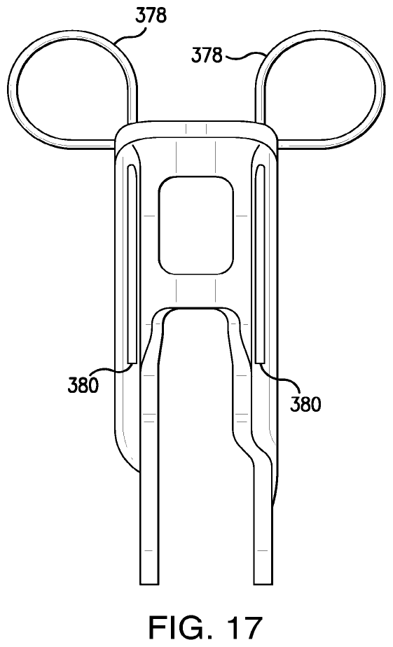

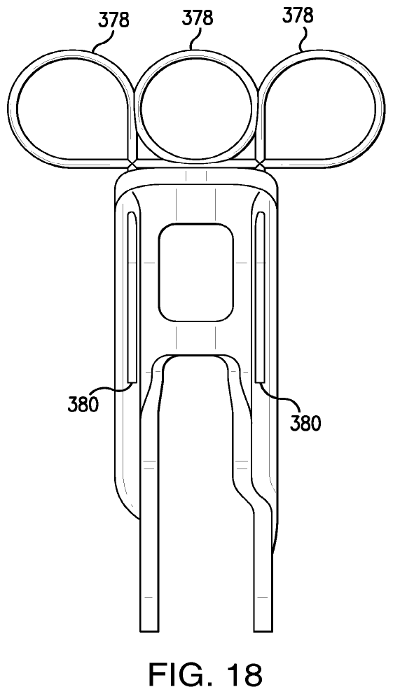

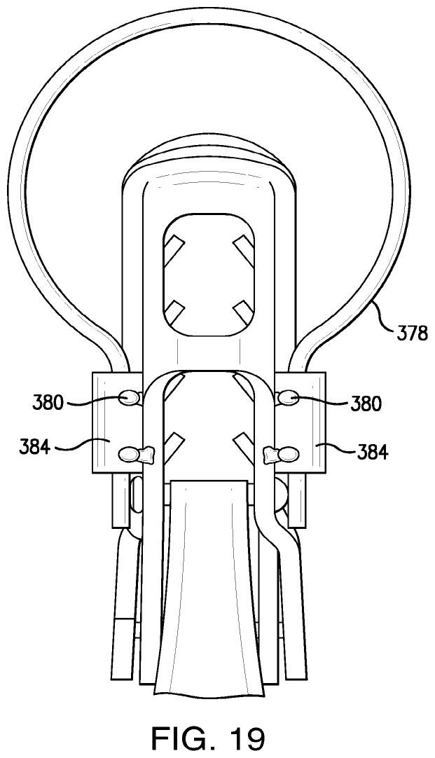

In accordance with the disclosed subject matter, the wire frame 246 can include at least one wire loop 378, such as the wire loop shown in the examples of FIGS. 14 and 19. Such wire loop 378 may extend laterally outwardly from a first location 380 on arm 108 and distally beyond a second end 142 of arm 108 and back to a second location 380 on arm 108 such that wire loop 378 of wire frame 246 effectively increases the width and length of arm 108 while in an undeformed condition according to one example. The wire frame 246 can include at least two wire loops 378, as shown in the examples of FIGS. 15-17 and 20-23. Referring in addition to FIGS. 15 and 16, the at least two wire loops 378, just like the single wire loop 378 of FIG. 14, can be configured to fold or deform inwardly to fit within an inner diameter of an interventional catheter 402 in various examples. As the size adapter 200 exits the distal end of the interventional catheter, the size adapter 200 can then expand to the undeformed maximum size adapter width 202. In some embodiments, the inner surface of the interventional catheter can restrain the size adapter 200 in the deformed condition. To increase the contact patch area, as discussed above, the maximum size adapter width 202 thus can be greater than the inner diameter of the interventional catheter. The size adapter 200 can have a variety of configurations to transition from the deformed condition to the undeformed condition, such as, but not limited to, the various configurations disclosed herein. As shown in the example of FIG. 18, the wire frame 246 can include at least three wire loops 378. Such wire loops 378, just like the two-wire loop configuration of FIG. 17, in the nondeformed condition may be substantially positioned distal of the second end 142 of arm 108 and may not only increase the effective width of arm 108 but also its length. In other words, in the nondeformed or undeformed condition, second end 147 of arm 108 is positioned between wire loops 378 of FIGS. 17 and 18 and first end 140 of arm 108.

With additional reference to FIGS. 19, 21, and 23, a tubing attachment 384 can be welded to the at least one arm 108, and the wire frame 246 can be inserted into the tubing attachment 384 in some examples. During manufacturing, the tubing attachment 384 can be laser welded to the body lateral side 166, for example. The tubing attachment can enable the wire to slide and/or rotate within the tubing which can vary the coaptation width and/or allow for easier deployment. In other words, the opposed ends of wire frame 246 may be moveable relative to arm 108 such that an effective length of wire frame extending from tubing attachments 284 may be selectively controlled to vary the increase in length and width of arm 108 provided by wire frame 246.

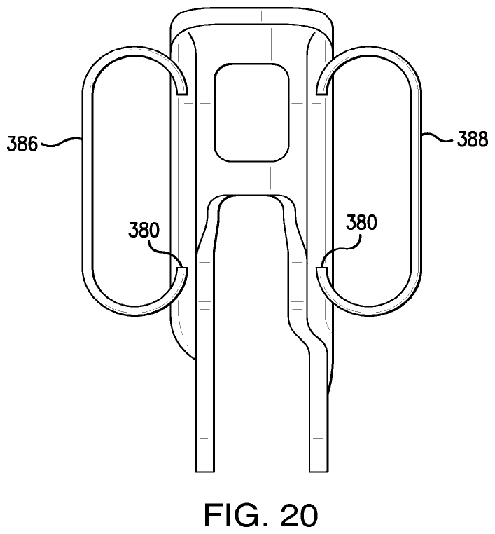

As shown in FIGS. 20-23 for purpose of illustration and not limitation, the wire frame 246 can include a first wire frame 386 and a second wire frame 388, wherein the first wire frame 386 is separate from the second wire frame 388 in some examples. Furthermore, in various examples, the wire frame 246 can include a plurality of separate wires 390 grouped together which forms a plurality of overlapping or stacked loops, as illustrated in FIGS. 22 and 23. Each of the plurality of wires 390 can be welded, for example, to the arm 108, as illustrated in FIGS. 20 and 22. For example, first and second ends of each of the plurality of wires 390 may be welded to arm 108. Multiple loops of small wire can increase the flexibility of the first and second wire frame 386, 388.

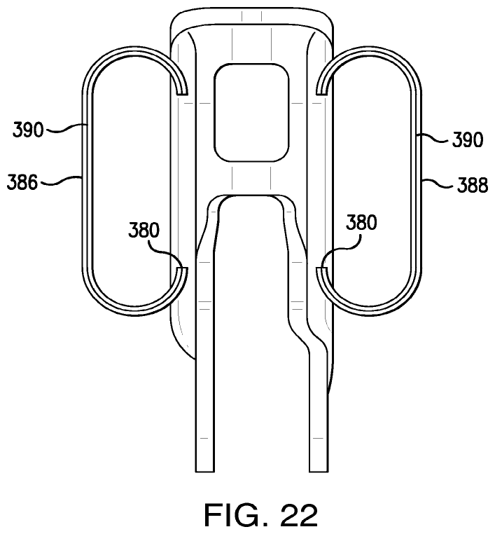

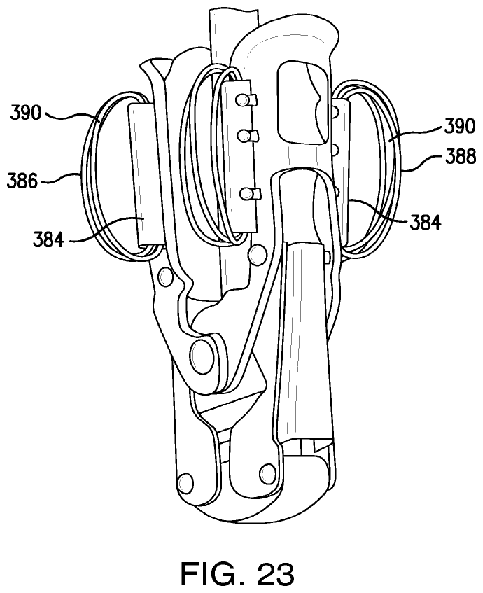

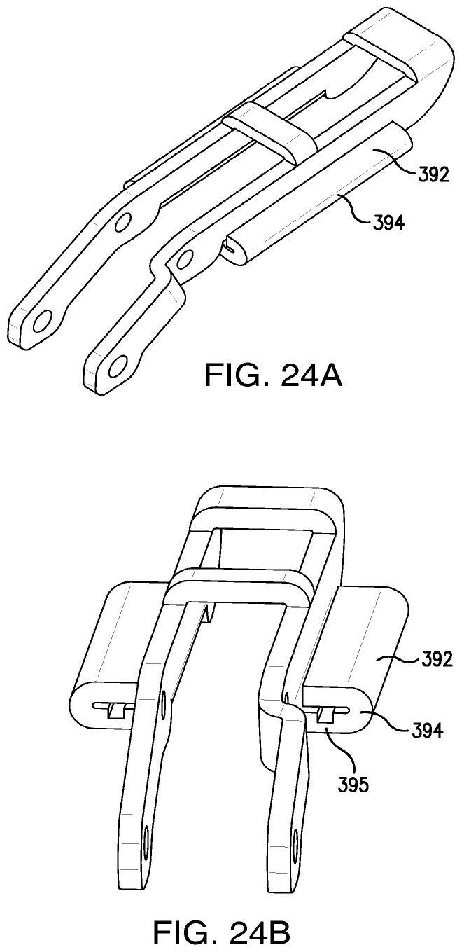

Further in accordance with the disclosed subject matter, and as shown in FIGS. 24A and 24B, examples of the fixation device can further include an attachment wing 392 extending laterally from the at least one arm 108, wherein the attachment wing 392 and the at least one arm 108 can be a single piece structure, and further wherein a portion of the wire frame 246 can be attached to the attachment wing 392. In one example, the attachment wing 392 can be formed of a sheet metal fold 394 comprising sheet metal folded 180 degrees around the portion of the wire frame 246. The attachment wing 392 can be machined with a coining process, for example. The attachment wing 392 can, for example, include a machined slot 395 sized for the portion of the wire frame 246, as shown in FIG. 24B. The slot 395 can be located in a hem or seam of the attachment wing and may extend into the attachment wing 392 perpendicular relative to the hem according to various examples of the disclosure. The slot 395 in the hem can control wire rotation according to various examples. The attachment wing 392 can be press fit around the portion of the wire frame 246, for example. The attachment wing 392 can be stamped or press fit around the portion of the wire frame 246 in various examples.

In accordance with another aspect of the disclosed subject matter, the fixation device can, in some examples, include a gusset member 396 disposed between the body portion 138 and the first nondeformable wing extension 352. In this regard, gusset member 396 may provide structural reinforcement between body portion 138 and nondeformable wing extension 352. The gusset member 396 can include a hole 397, and the size adapter 200 may be attached to the hole 397 in one example. Such attachment may be a slidable attachment or a fixedly secure attachment. Additionally, hole 397 may be parallel to the longitudinal axis of the arm 108 in one example.

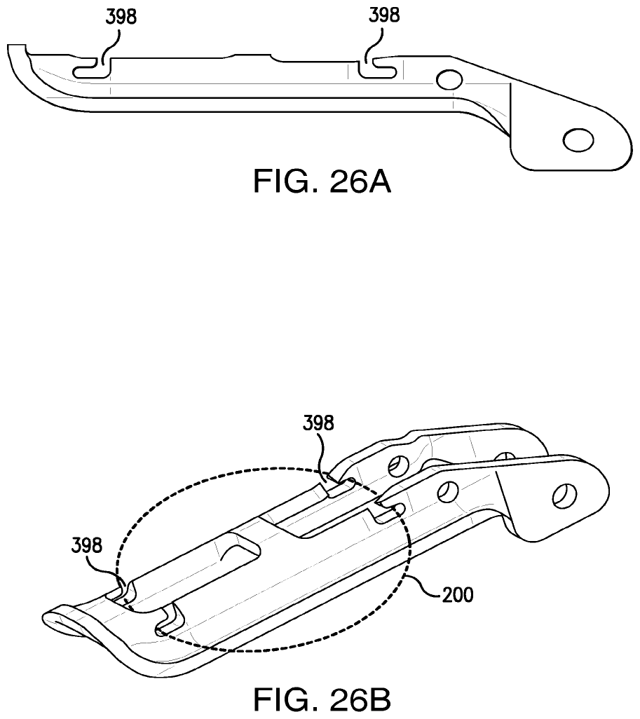

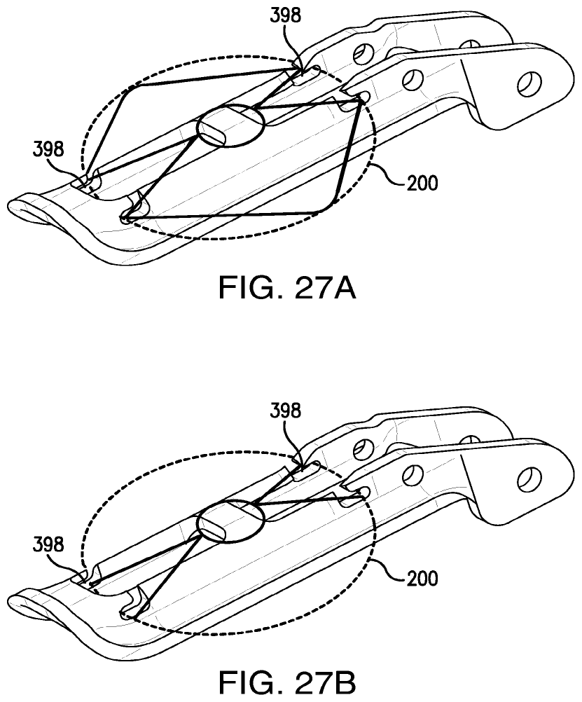

Referring now in addition to FIGS. 27A-27D, the body portion 138 can, in one example, include at least one notch 398, and the size adapter 200 can be attached to the at least one notch 398 by a snap fit. The at least one notch 398 can be is machined into the body portion and a wire loop can be pressed into place with int the notch 398 in various examples. More specifically, arm 108 may have pairs of notches 398 formed in the back surface 304 thereof at opposite sides of longitudinal axis 156. In this regard, a first and second notch 398 of a notch pair may first extend into back surface 304 and toward the front surface 302 of arm 108 and then extend axially in opposition directions from each other such that a size adapter 200 in the form of a wire frame ring, like that shown in FIG. 26B, may be expanded within such notches 398 into a snap fit arrangement within notches 398 and retained therein. Additionally, support struts, like those shown in FIGS. 27A-27B may provide expandable support to the wire frame ring 200 in some examples.

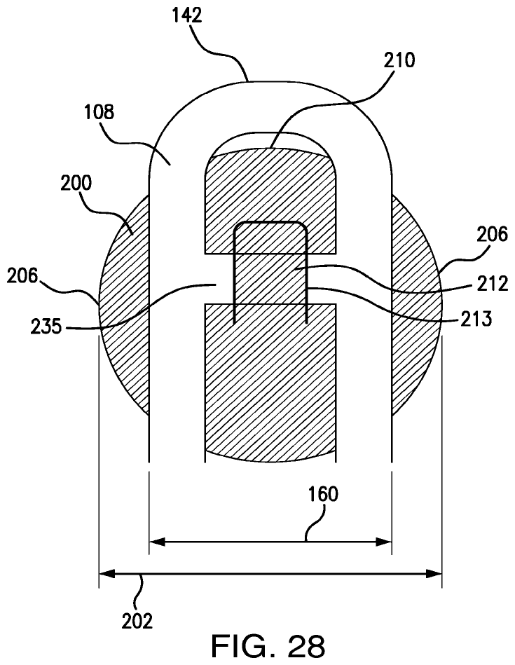

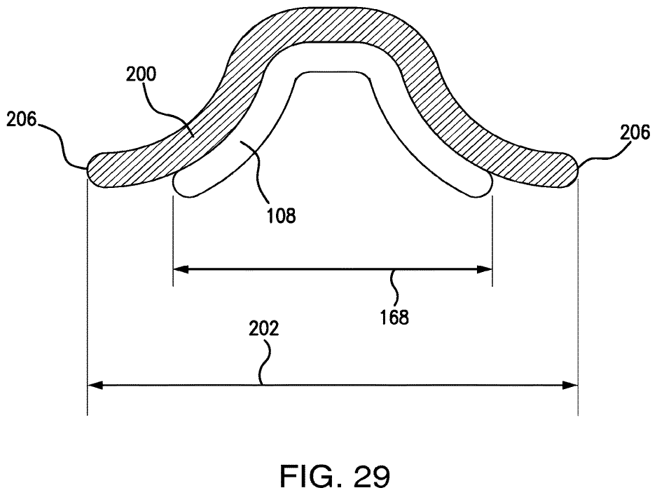

With additional reference to FIGS. 28 and 29, the at least one arm 108 can include a strut member 235 extending perpendicular to the longitudinal axis 156, and wherein the size adapter 200 can be attached to the strut member 235 in various examples. The size adapter 200 can be attached to the at least one arm 108 with a flap member 212 and/or a suture 213, for example. As shown in FIG. 29, the size adapter may conform to the shape of the backside 304 of arm 108 while extending beyond its lateral edges in one example.

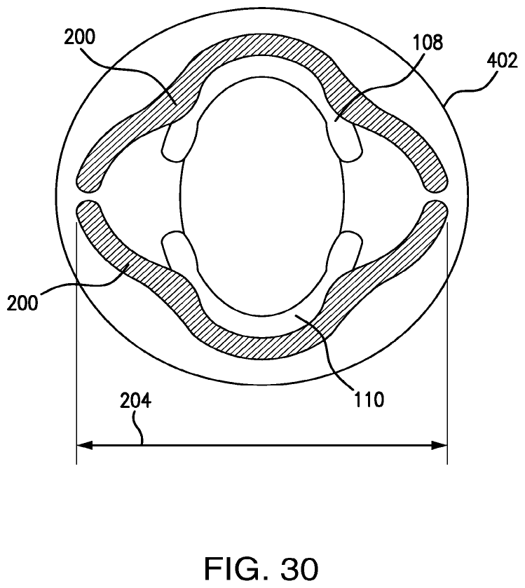

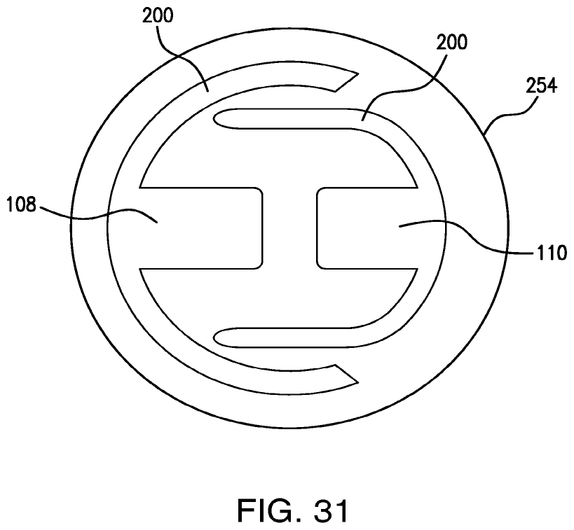

As depicted in FIG. 30, when the fixation device 104 is in the interventional catheter, the size adapter 200 of the first arm 108 can be next to the size adapter 200 of the second arm 110, without any overlap between the respective size adapters 200 in one example. Alternatively, and as depicted in FIG. 31, the size adapter of the first arm 108 can overlap with the size adapter of the second arm 110 when in the interventional catheter such that a portion of the size adapter 200 of the second arm 110 is nested within a portion of the size adapter 200 of the first arm 108 in one example. This overlapping configuration can accommodate wider maximum size adapter width dimensions 202, while still being deliverable in the interventional catheter.

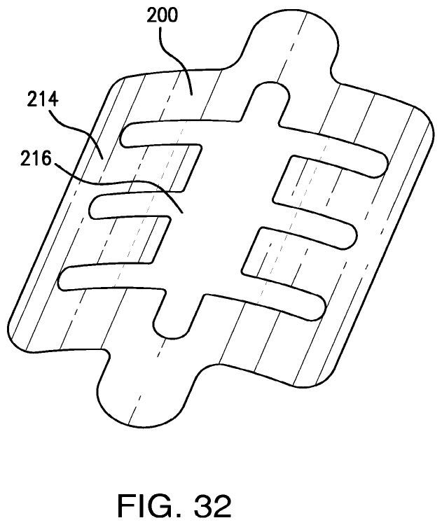

In accordance with another aspect of the disclosed subject matter, and as shown in FIG. 32, the size adapter 200 can have a deformable portion 214 and at least one reinforced portion 216, wherein the at least one reinforced portion 216 can be less flexible than the deformable portion 214 in various examples. The reinforced portion 216 can be disposed along a center of the size adapter aligned with the longitudinal axis 156 of the arm such as to form a spine portion. The at least one reinforced portion can also include at least one traverse reinforced portion 216 extending from the spine portion and aligned perpendicularly to the longitudinal axis 156 of the at least one arm 108, for example. It is understood the reinforced portion 216 can be configured in any number of ways to provide a desired degree of rigidity at desired locations on the size adapter 200. The deformable portion 214 can comprise Nitinol and additionally or alternatively a soft plastic in various examples. In an alternative embodiment, the deformable portion 214 can, for example, be tapered at the size adapter second end 210 to facilitate moving size adapter 200 into the deformed condition when the fixation device is retracted into the catheter.

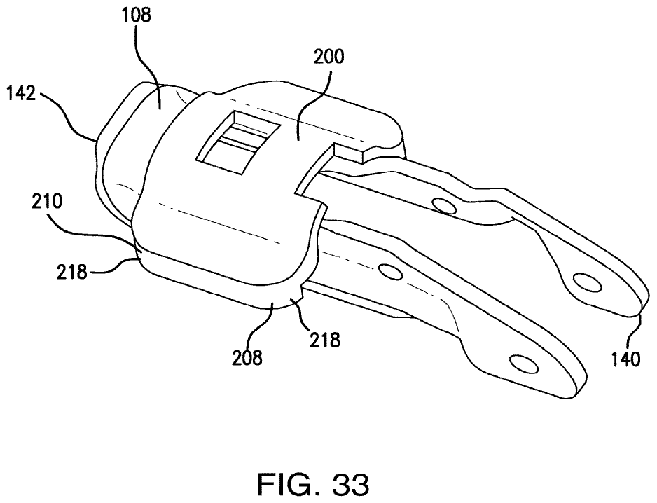

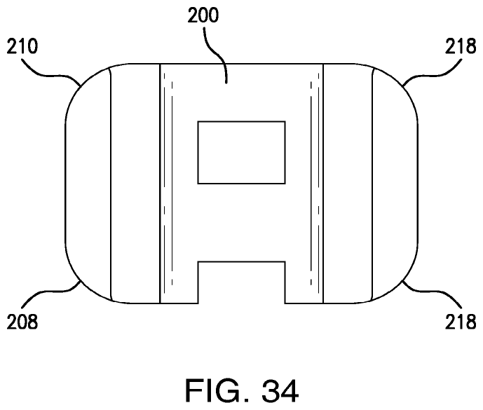

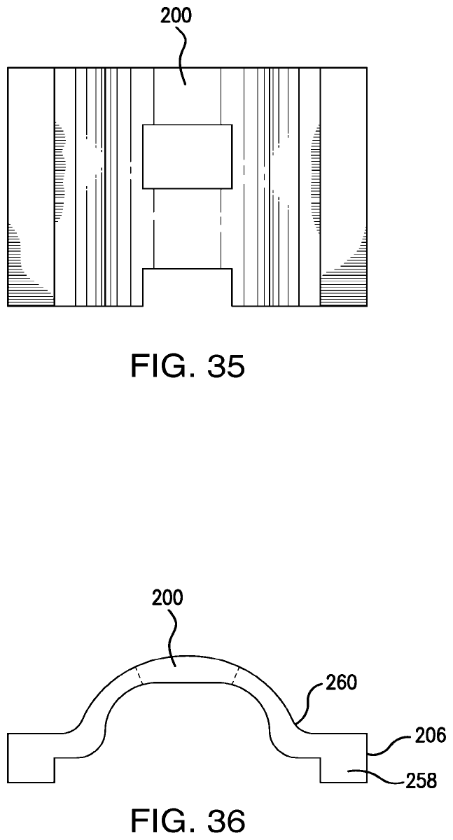

For purpose of illustration and not limitation, the example size adapter 200, as shown in FIGS. 33-34, can have a first end 208 disposed proximate the second end 142 of the at least one arm 108, and wherein the second end 210 of the size adapter 200 can include a radiused or rounded corner 218. The radiused corner on the second end 210 can facilitate retraction of the size adapter back into the catheter, if desired during a procedure. Likewise, the size adapter 200 can have a first end 208 disposed closer to the first end 140 of the at least one arm 108 than the second end 142 of the at least one arm 108, and wherein the first end 208 of the size adapter 200 can include a radiused corner 218, for example. The radiused corner on the first end 208 can facilitate delivering the size adapter through a tortious catheter path. It is understood that any number of other corner configurations can be used, for example tapered corners. FIGS. 34 and 35 illustrate an exemplary plan view of a size adapter 200 wherein FIG. 34 includes radiused corners 218 and, for purpose of comparison, FIG. 35 does not include radiused corners 218.

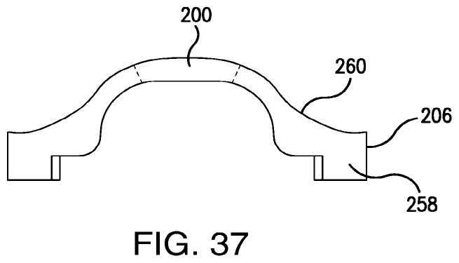

Referring now in addition to FIGS. 36 and 37, which illustrate an exemplary end view of the size adapter 200 depicted in FIG. 34. In the examples of both FIGS. 36 and 37 the size adapter 200 can include a lateral section 258 disposed proximate the outer lateral edges 206 of the size adapter 200. The lateral section 258 can have an increased thickness in one example. With continued reference to FIGS. 36 and 37 each exemplary size adapter 200 can include a fillet portion 260 disposed on a back surface of the size adapter.

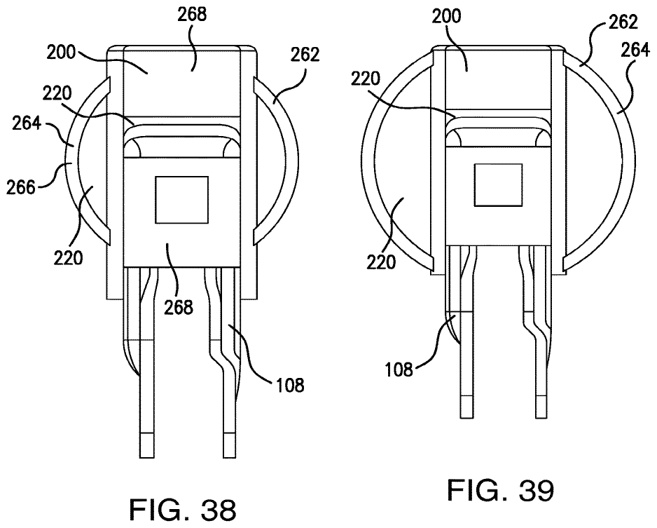

Referring in addition to FIGS. 38-43, the size adapter 200 can extend beyond the second end 142 of the body portion 138 in some examples. The size adapter 200 can include at least one opening 220 aligned with the second end 142 of the body portion 138 in various examples. The size adapter 200 can, for example, include a deformable frame 262 having at least one deformable flex portion 266, wherein the at least one deformable flex portion 266 includes a wire element 264 defining an opening 220 therethrough in plan view. The wire element 264 can extend beyond the second end 142 of the body portion 138.

The deformable frame 262 can be attached to the at least one arm 108 by a molded structure 268 in some examples. The molded structure 268 can extend beyond the second end 142 of the body portion 138, as shown by FIGS. 38-40, for example. However, while the molded structure 268 may extend beyond the second end 142 of the body portion 138 of arm 108, flex portion 266 may not extend beyond an end of the molded structure 268 in some examples.

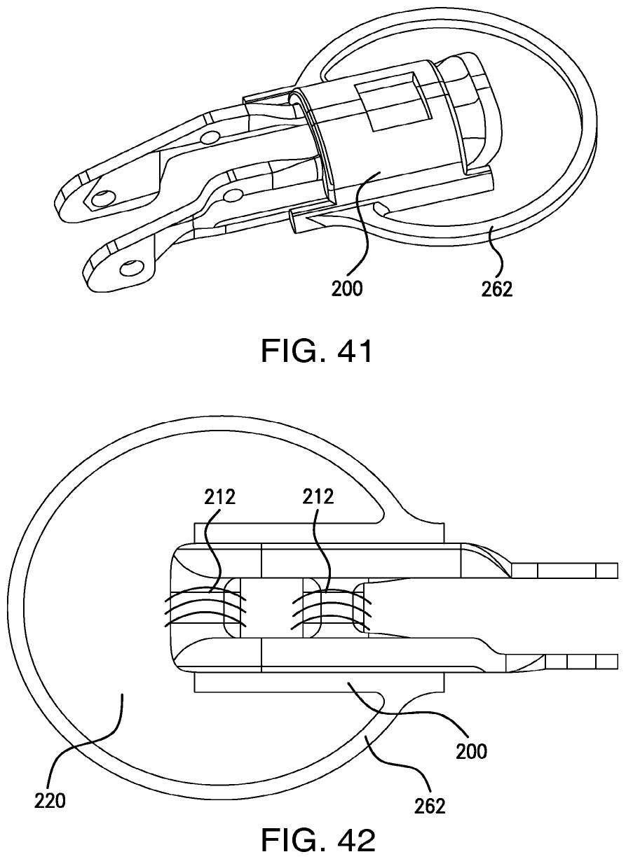

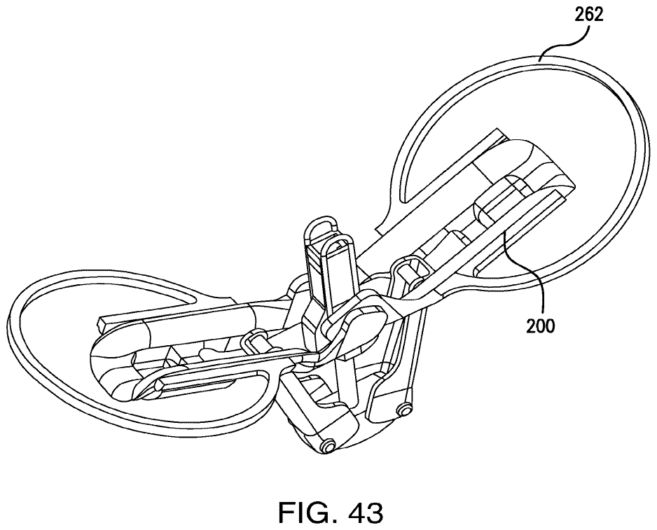

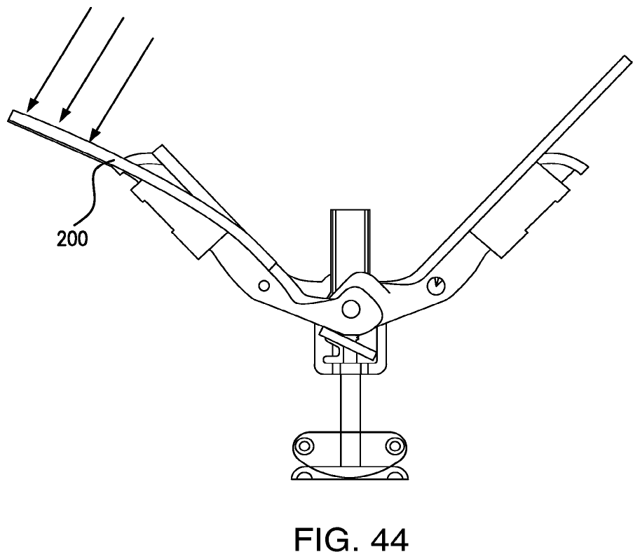

In accordance with the disclosed subject matter, FIGS. 41-43 depict an alternative embodiment of an alternative embodiment of size adapter 200 including a single deformable frame 262 that extends from one side of the body portion 138, to beyond the second end 142 of the body portion 138, and returns to the opposing side of the body portion 138, for example. The exemplary size adapter 200 can, for example, have an attachment portion that attaches to and generally conforms to the back surface 308 of arm 108 and a saucer shaped tip component extending from the attachment portion and defining an opening 220 with a diameter of between about 0.23 inch and 0.47 inch, and preferably between about 0.31 inch and 0.39 inch, although other suitable diameters can be used. Thus, this embodiment is similar to that of FIG. 14 with the exception that the saucer shaped tip is secured to the attachment portion of deformable frame 262 that itself attaches to arm 108 rather than the saucer shaped tip being directly secured to arm 108. Such attachment portion may be secured or additionally secured to arm 108 by a plurality of wires or sutures 212 as shown in FIG. 42, for example. FIG. 44 illustrates a front view of the fixation device of FIG. 43 with the size adapter 200 undergoing a load proximate the illustrated arrows at the tip of the size adapter, wherein the load is about 0.14 lbf leaflet load during the cardiac cycle. As depicted, the exemplary size can deflect under sufficient load to a non-parallel orientation relative the longitudinal axis of the corresponding arm.

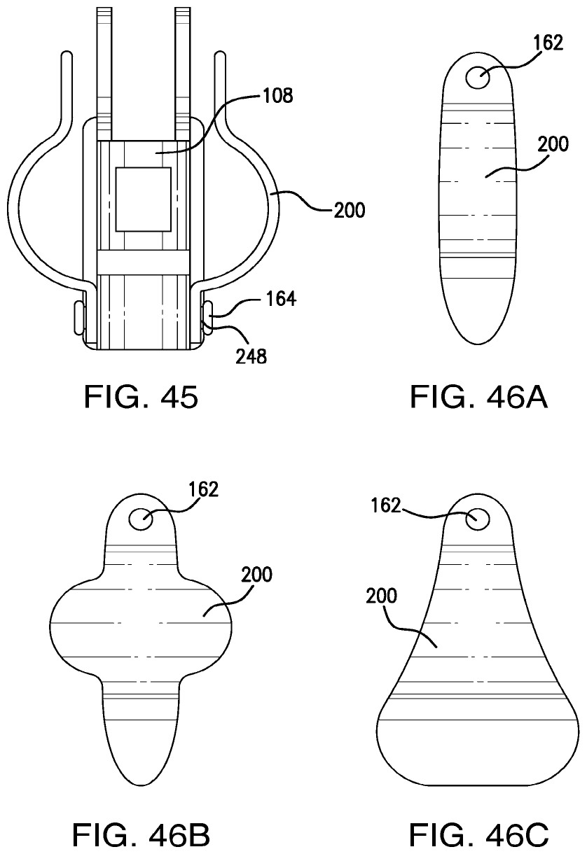

With additional reference to FIG. 45, the example size adapter 200 can be configured to pivot relative to the at least one arm 108 at a pin connection 248. For example, the pin connection 248 can be a rivet 164. Further, size adapter 200 can be configured to pivot relative to the at least one arm 108 member at the pin connection 248 in one example. As embodied herein, the pin connection 248 can include two connection points disposed on each side of the opposing body lateral sides 166 of the at least one arm 108. Fixation devices attached by the pin connection 248 can be of various shapes and sized, as shown in the examples of FIGS. 46A-46C. In preferred embodiments, size adapters 200 shown in FIG. 46A through FIG. 46C are aligned with the arm axis 156 such that rotation about pin joint 162 is prevented or resisted with a spring. Additionally, size adapters shown in FIG. 46A through FIG. 46C have curved and flexible mid-sections that can be flatted and/or elongated to a lower profile during device delivery through a hollow sheath or tubular guide catheter according to various examples of the disclosure. In accordance with another aspect of the disclosed subject matter, the size adapter 200 can have a rivet 366 attachment to the back of the arm 108 proximate the trough 145, for example.

With additional reference to FIG. 47, a pair of example size adapters 200, each in the form of a wire frame, may extend laterally from body portion 138 of arm 108 and may each partially define an opening 220 between body portion 138 and adapter 200. One or both ends 366 of each size adapter 200 may be slidingly attached to side slots within body portion 138 such that laterally inward compression of each size adapter 200 causes size adapters 200 to lengthen and one or both ends 266 to slide within their respective slots, for example. Similarly, decompression of each size adapter 200 from the retracted/compressed condition causes the ends 366 to slide within the slots such that the size adapters 200 shorten and increase their effective lateral width under the natural bias of size adapters 200 according to examples of the disclosure.

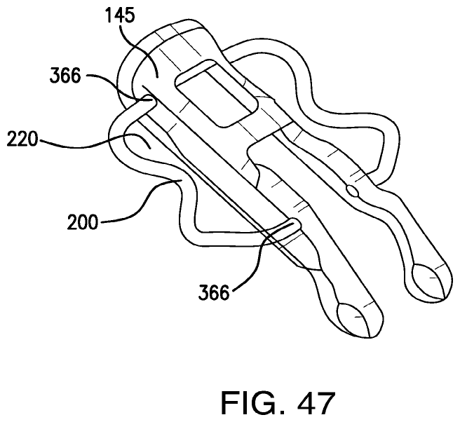

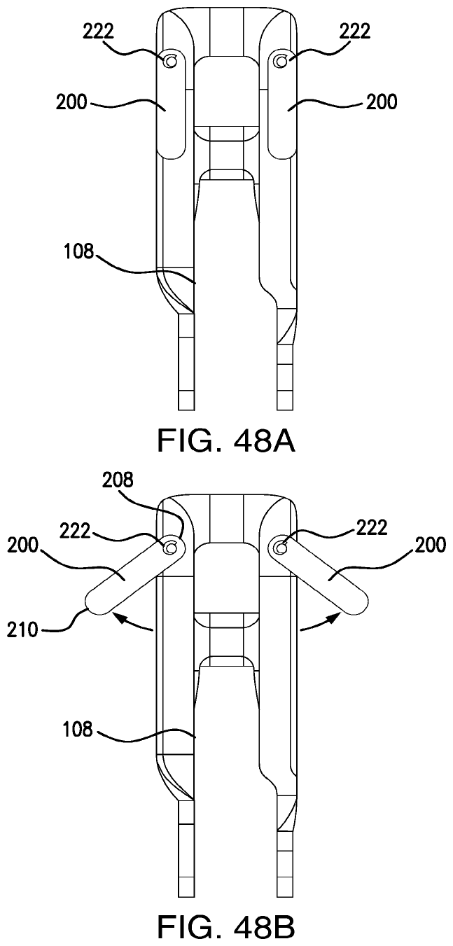

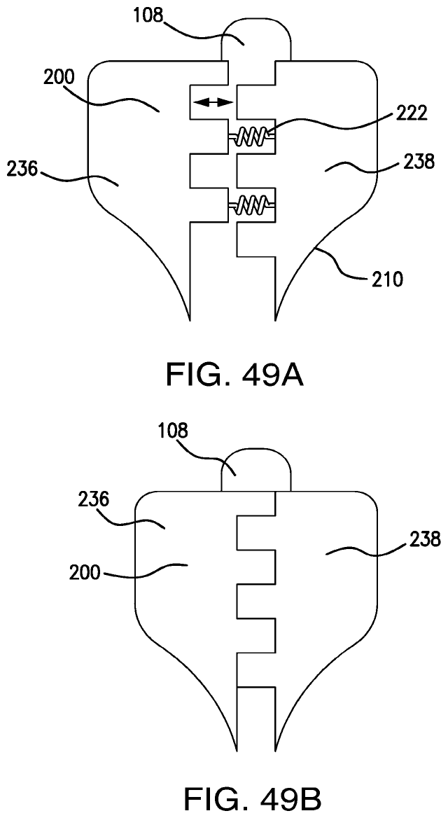

With further reference to FIGS. 48A-49B, the size adapter 200 can include at least one spring 222 configured to bias the size adapter 200 towards the maximum undeformed arm lateral cross-dimension 202, for example. With reference to FIGS. 48A-48B, the size adapter 200 can, for example, include an elongate member pivotally attached at a first end 208 to the at least one arm 108 and extending to a second end 210 that is a free end wherein the at least one spring 222 can be configured to bias the second end 210 laterally outwardly from the at least one arm 108. With reference to FIGS. 49A-49B, the size adapter 200 can, for example, include a first section 236 and a second section 238 with springs 222 disposed between the first section 236 and the second section 238 configured to separate the first section 236 and the second section 238 from each other towards the maximum undeformed arm lateral cross-dimension 202. Such springs 222 may be torsion springs, as in the embodiment of FIGS. 48A-48B, or axial compression springs, as in the embodiment of FIGS. 48A-49B. In this regard, torsion springs 222 may cause the elongate members to rotate from a first retracted condition to a second deployed condition. On the other hand, axial compression springs 222 may translationally move first and second sections 236, 238 from a first retracted condition to a second deployed condition. Prior to a procedure, crimping can be used to get the device into an introducer for insertion into the inner surface of the interventional catheter. As embodied herein, the multiple sections 236, 238 can, for example, have an angled profile or taper at the second end 210 to allow the size adapter 200 to deform if the fixation device 104 is retracted back into the catheter.

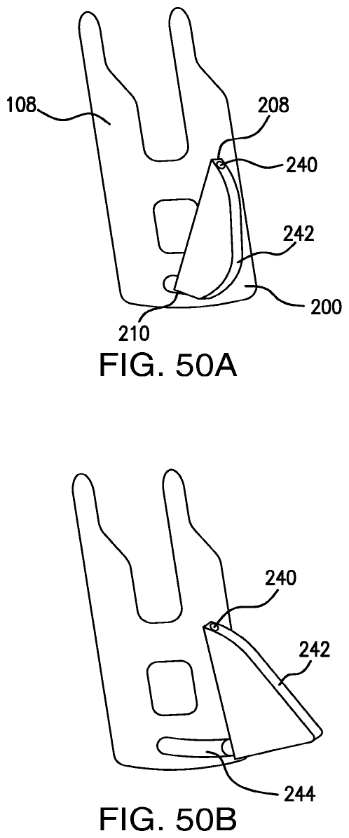

With additional reference to FIGS. 50A-50B, these figures depict an example of a size adapter 200 that includes a pivoting wing 242. The pivoting wing 242 may, for example, be pivotably attached to arm 108 via a pivot pin 240 at its first end 208 and slidingly attached to a track or groove 244 within arm 108 at its second end 210. Wing 242 may be pivotable along track 244 from a first retracted condition (see FIG. 50A) to a second deployed condition (see FIG. 50B), for example. A spring or other biasing mechanism (not shown) may bias wing 242 to the deployed position.

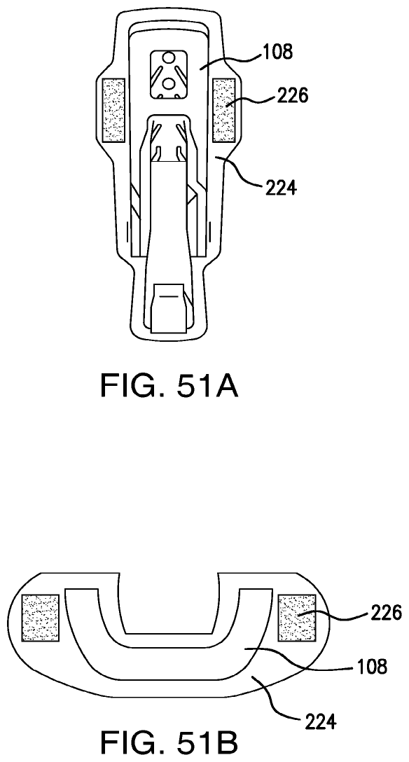

With additional reference to FIGS. 51A-51B, the size adapter 200 can, for example, include an expandable member 226 configured to receive fluid such that, in an expanded condition, the expandable member 226 has more fluid in the expanded condition than in an unexpanded condition. The expandable section 226 can be a fillable section or an absorbable section according to various examples. The expandable member 226 can be disposed proximate the body lateral side 166, for example. The expandable member 226 and the arm 108 can be surrounded by a cover 224 in one example, he cover 224 can attach the expandable member 226 to the arm 108. Additionally or alternatively, it is understood that any number of other fasteners can be used, either alone or in combination with the cover 224.

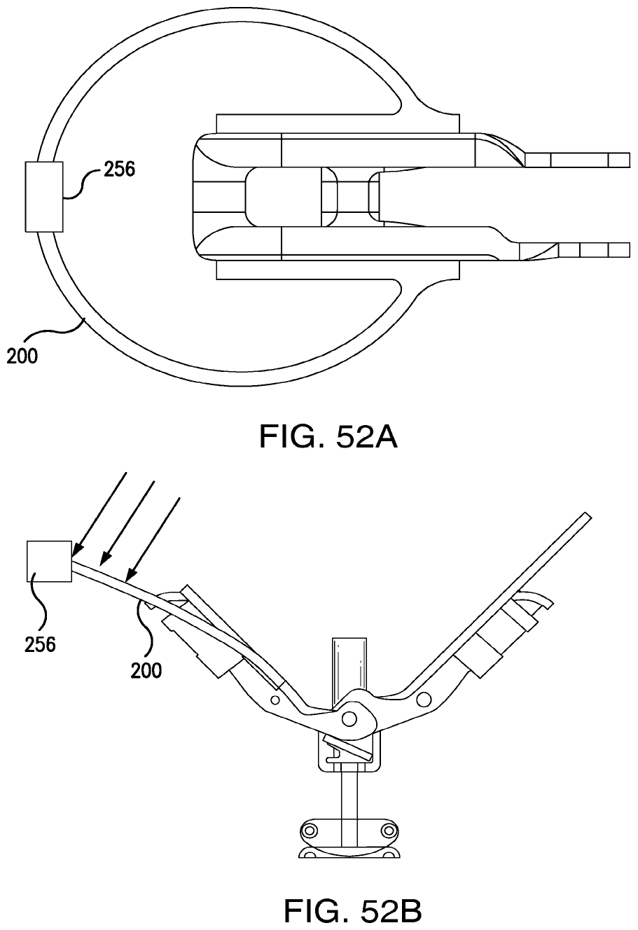

As shown in FIGS. 52A-52B and in accordance with the disclosed subject matter, a size adapter 200, such as any of the various size adapters disclosed herein, can include at least one radiopaque marker 256. The radiopaque marker 256 can be disposed at any location on the size adapter 200, for example proximate an end or tip of the size adapter 200, as depicted. The radiopaque marker 256 can allow a user to obtain a visual cue on X-ray fluoroscopy of the amount of tension being applied to a leaflet, as well as how cyclic the tension is occurring in some examples. For example, if a user does not see deflection of the radiopaque marker 256, then this can indicate that an insufficient amount of tissue is being grasped and a re-grasping process may be required. Similarly, if a user sees excessive static (stable) deflection of the radiopaque marker 256, this may indicate that too much tension has been placed on the leaflet in a particular area. Furthermore, if a user sees more deflection on one size adapter 200 as compared to another corresponding size adapter 200, this can indicate uneven leaflet insertion which can prompt a re-grasp procedure, or this can show that the catheter is applying an uneven side load. In accordance with the disclosed subject matter, the radiopaque marker 256 can be secured to the size adapter 200 with any number of fixtures including a suture or press fit. For illustration and not limitation, the size adapter 200 with a radiopaque marker 256 depicted in FIG. 52B is undergoing a load at the illustrated arrows of about 0.14 lbf leaflet load during cardiac cycle, and the tip of the size adapter with a radiopaque marker 256 is depicted in a deflected condition.

The arms 108, 110 of fixation device 104 may, for example, be symmetrically configured with any of the aforementioned sizer adapter embodiments. In this regard, first and second arms 108, 110 of fixation device 104 may each include one or more of the same size adapters 200, for example. However, other embodiments of fixation device 104 may be asymmetrically configured such that the first arm 108 may include one or more size adapters 200, while the second arm 110 may not include a size adapter 200. Further, asymmetry may be provided by utilizing one or more of the aforementioned size adapters 200 on the first arm 108, while utilizing a different size adapter 200 on another arm. As such, fixation device 104 can be asymmetrically configured with size adapters 200 so that one arm 108, 110 has an effective width and/or length greater than the other arm 108, 110 to optimally address various circumstances such as a prolapsing leaflet, a wide jet originating from one leaflet, a short or restricted leaflet, and narrow grasping regions, for example.

As previously noted, and in accordance with the disclosed subject matter, examples of the fixation device 104 further include at least one gripping element 116, for example, the first gripping element 116 and second gripping element 118 as shown in FIG. 1. The gripping element 116 can, for example be moveable relative to the at least one arm 108 to capture a native leaflet therebetween. In particular, the at least one gripping element 116 has a first end 228 coupled to a portion of the fixation device and a second end 230 moveable relative to the at least one arm 108. In accordance with the disclosed subject matter, each arm 108 can, for example, be configured to define or have a trough 145 aligned along the longitudinal axis 156. The trough 145 can be configured to receive the gripping element 116 therein.

As embodied herein, in one example, each gripping element includes a plurality of friction elements 152, such as in rows. For example, each gripping element 116, 118 can have at least four rows of friction elements 152. The friction elements 152 can allow for improved tissue engagement during leaflet capture. If the fixation device requires adjustment after an initial leaflet capture, the arms can be opened, the gripping element can be raised vertically, and tissue can disengage from the fixation device 104, facilitating re-grasp and capture.

For example, and with reference again to FIG. 1, and as further embodied herein, each gripping element 116, 118 can be biased toward each respective arm 108, 110. Prior to leaflet capture, each gripping element 116, 118 can be moved inwardly toward a longitudinal center of the device (i.e., away from each respective arm 108, 110) and held with the aid of one or more gripping element lines (not shown) which can be in the form of sutures, wires, rods, cables, polymeric lines, or other suitable structures. The gripping line elements can be operatively connected with the gripping elements 116, 118 in a variety of ways, such as by being threaded through loops (not shown) disposed on the gripping elements 116, 118, for example.

For each embodiment disclosed herein, the fixation device can further include an assembly to move the arms between various defined positions, for example, and not limitation, and with reference to FIG. 1., the fixation device 104 embodied herein includes two link members or legs 306, each leg 306 having a first end which is rotatably joined with one of the arms 108, 110 and a second end which is rotatably joined with a base 170. The base 170 can be operatively connected with a stud 176 which can be operatively attached to a distal end of a delivery shaft (not shown for clarity). In some embodiments, the stud 176 can be threaded so that the distal end of a delivery shaft can attach to the stud 176 by a screw-type action. Further, the connection point between the stud 176 and the distal end of a delivery shaft can be disposed within the coupling member 174. However, the distal end of a delivery shaft and stud 176 can be operatively connected by any mechanism which is releasable to allow the fixation device 104 to be detached. The stud can be axially extendable and retractable to move the base and therefore the legs 306 which rotate the arms 108, 110 between closed, open and inverted positions. Likewise, immobilization of the stud 176, such as by a locking mechanism 178, can hold the legs 306 in place and therefore lock the arms 108, 110 in a desired position, for example. Further details are disclosed in the patents and publications incorporated by reference herein.

The embodiments illustrated herein are adapted for repair of a heart valve, such as a mitral valve, using an antegrade approach from a patient's left atrium, and a tricuspid valve, using an antegrade approach from a patient's right atrium. Prior to a procedure, imaging and various tests can be performed to anticipate and diagnose a patient's individual circumstances and assist a physician in selecting a fixation device with components, such as the size adapter 200, having the desired parameters. Indeed, a physician can select a desired fixation device from a plurality of fixation devices having varied parameters and features. Further, after imaging a patient, a physician can configure a selected fixation device with desired components, such as a desired size adapter 200. Alternatively, various components, such as the size adapter 200 can be attached to the at least one arm during the manufacturing process.

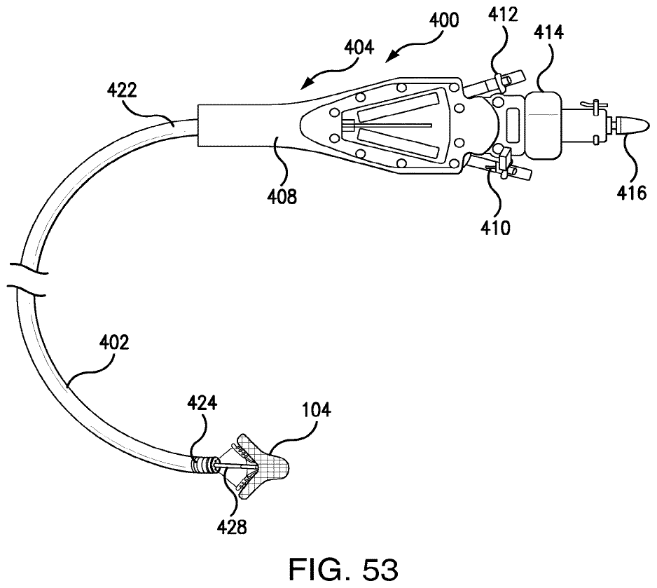

Referring in addition to FIG. 53 for purpose of illustration and not limitation, an exemplary interventional catheter assembly 400 is provided for delivery of the fixation device 104. That is, the interventional catheter assembly 400 can be used to introduce and position a fixation device (e.g., fixation device 104). The interventional catheter assembly 400 can, in this example, include an interventional catheter 402, having a proximal end portion 422 and a distal end portion 424, and a handle 404 attached to the proximal end portion 422. A fixation device 104 can be removably coupleable to the distal end portion 424 for delivery to a site within the body, for example, the mitral valve or the tricuspid valve. Extending from the distal end portion 424 is actuator rod 428. The actuator rod 428 is connectable with the fixation device 104 and can act to manipulate the fixation device 104, for example, opening and closing the arms. Handle 404 of the interventional catheter assembly 400 is shown, including main body 408, gripping element line handle 412, lock line handle 410, actuator rod control 414, and actuator rod handle 416, among other features.

During a procedure, to access a valve, such as a mitral valve or a tricuspid valve, the interventional catheter 402 can be inserted from a puncture in the femoral vein, through the inferior vena cava and into the right atrium. For access to the mitral valve, the interventional catheter 402 can extend through a puncture in a fossa of the interatrial septum and curve so that the distal end portion 424 is directed over the mitral valve. For access to the tricuspid valve, the interventional catheter 402 can curve in the right atrium so that the distal end portion 424 is directed over the tricuspid valve. For any valve, the distal end portion 424 can be centered over an opening between the leaflets of the valve. The distal end portion 424 can be lowered into the valve, thereby lowering the fixation device into the ventricle. The distal end portion can be raised and lowered as desired for a procedure, such as a regurgitation correction procedure. Prior to a procedure, imaging and various tests can be performed to anticipate and diagnose a patient's individual circumstances and assist a physician in selecting a fixation device having the desired parameters.

While the embodiments disclosed herein utilize a push-to-open, pull-to-close mechanism for opening and closing arms it should be understood that other suitable mechanisms can be used, such as a pull-to-open, push-to-close mechanism. Likewise, other actuation elements can be used for deployment of the gripping elements.

Although the subject matter disclosed herein has been described with reference to particular embodiments, it is to be understood that these embodiments are merely illustrative of the principles and applications exemplified by such embodiments. It is therefore to be understood that numerous modifications may be made to the exemplary embodiments and that other arrangements may be devised such as combining one or more features of one embodiment with another embodiment or features from a plurality of embodiments, as an example. Thus, the exemplary embodiments herein are not intended to be exhaustive or to limit the disclosed subject matter to those embodiments disclosed.

1. A fixation device for fixation of leaflets of a heart valve, the fixation device comprising:
a central assembly;
at least one arm moveably coupled to the central assembly, the at least one arm comprising a body portion having a first end and a second end and a longitudinal axis defined therebetween, the second end being moveable between a closed position and an open position, the body portion having opposing body lateral sides, each body lateral side extending between the first end and the second end, the body portion having a body portion width defined between the opposing body lateral sides;
a size adapter attached to the at least one arm, the size adapter having an undeformed condition in which a maximum undeformed arm lateral cross-dimension is defined between outer lateral edges of the size adapter and a deformed condition in which a maximum deformed arm lateral cross-dimension is less than the maximum undeformed arm lateral cross-dimension, wherein the ratio between the body portion width and the maximum undeformed arm lateral cross-dimension is at least about 1:1.8 to about 1:2.2; and
at least one gripping element moveable relative to the at least one arm to capture a native leaflet therebetween.

2. The fixation device of claim 1, further comprising first and second nondeformable wing extensions, each nondeformable wing extension extending laterally from a respective body lateral side, each nondeformable wing extension having a lateral outer edge, wherein a maximum wing width is defined between the lateral outer edge of the first nondeformable wing extension and the lateral outer edge of the second nondeformable wing extension.

3. The fixation device of claim 2, wherein the maximum wing width is between about 1.40 and 1.60 of the body portion width.

4. The fixation device of claim 2, wherein the size adapter is a first and second flexible attachment attached to the first and second nondeformable extension, respectively, wherein each flexible attachment comprises a flex portion and an attachment portion.

5. The fixation device of claim 4, wherein the ratio of the maximum deformed arm lateral cross-dimension to the maximum undeformed arm lateral cross-dimension is between 1:1 and 1.3:1.

6. The fixation device of claim 4, wherein the first and second flexible attachment are attached to the first and second nondeformable wing extension, respectively, by at least one fastener selected from the group consisting of a suture, weld, solder, snap, bolt, clamp, rivet, crimp, and adhesive.

7. The fixation device of claim 6, wherein the first flexible attachment is attached to the first nondeformable wing extension by at least two rivets, and the second flexible attachment is attached to the second nondeformable wing extension by at least two rivets.

8. The fixation device of claim 1, wherein the at least one arm has a front surface and a back surface, wherein the front surface faces the at least one gripping element when the at least one arm is in the closed position, and wherein the size adapter is disposed on the back surface of the at least one arm.

9. The fixation device of claim 8, wherein the size adapter is curved or bent to the front surface.

10. The fixation device of claim 4, wherein the first flexible attachment is connected to the second flexible attachment by a span that extends transverse to a longitudinal axis of the at least one arm.

11. The fixation device of claim 1, wherein the size adapter includes a material selected from the group consisting of: copper-zinc-aluminum alloy; copper-aluminum-nickel alloy; nickel-titanium alloy; nickel-titanium platinum alloy; and nickel-titanium palladium alloy.

12. The fixation device of claim 11, wherein the size adapter is formed of Nitinol.

13. The fixation device of claim 1, wherein the size adapter is a wire frame having a flexible structure.

14. The fixation device of claim 13, wherein the wire frame is secured to the at least one arm by at least one weld, wherein the at least one weld is disposed proximate one of the opposing body lateral sides.

15. The fixation device of claim 14, wherein each weld is disposed on a back surface of the arm, wherein the back surface of the body portion faces opposite the central assembly.

16. The fixation device of claim 13, wherein the wire frame comprises at least one wire loop.

17. The fixation device of claim 16, wherein the at least one wire loop extends from the at least one arm outwardly from respective first and second locations at the at least one arm and extends beyond the second end of the at least one arm.

18. The fixation device of claim 16, wherein the wire frame comprises at least two wire loops.

19. The fixation device of claim 18, wherein the at least two wire loops are configured to fold inwardly to fit within an inner diameter of an interventional catheter.

20. The fixation device of claim 18, wherein the second end of the at least one arm is positioned between the at least two wire loops and the first end of the at least one arm.

21-46. (canceled)
